(19) United States
(12) Patent Application Publication
(10) Pub. No.: US 2014/0234930 A1
(43) Pub. Date: Aug. 21, 2014

(54) SORGHUM WITH INCREASED SUCROSE PURITY

(71) Applicant: Michael F. Portereiko, Thousand Oaks, CA (US)

(72) Inventor: Michael F. Portereiko, Thousand Oaks, CA (US)

(21) Appl. No.: 14/126,620

(22) PCT Filed: Jun. 15, 2012
(86) PCT No.: PCT/US2012/042794

§ 371 Date: Dec. 16, 2013

Related U.S. Application Data
(60) Provisional application No. 61/497,610, filed on Jun. 16, 2011.

(51) Int. Cl. C12N 15/82; C12P 7/06

(52) U.S. Cl. CPC C12N 15/8246; C12N 15/8289; C12P 7/06
USPC 435/161; 800/303; 800/320

(57) ABSTRACT

The invention relates to materials and methods for increasing the sucrose purity and total sugar content in stalks of sorghum plants at maturity. The methods involve an inbred or an F1 hybrid transgenic sorghum plant containing transgenes that affect developmental stages such as spikelet meristem identity, establishment of floral meristem identity, or floral organ initiation, development, or function.

### CROSS-REFERENCE TO RELATED APPLICATIONS

This application claims priority to U.S. Provisional Application No. 61/497,610, filed Jun. 16, 2011, the disclosure of which is incorporated herein by reference in its entirety.

### TECHNICAL FIELD

The invention relates to sorghum plants with an increased total sugar and sucrose purity. In particular, the invention relates to sorghum plants with an increased total sugar and sucrose purity in the stalks at maturity, and methods and materials for making the same.

### BACKGROUND

Sorghum bicolor (Sorghum) is a cane and cereal species native to Africa that has many diverse cultivated, weedy, and wild variants. The canes of sweet sorghum are pressed for juice and fermented to fuel or used to make molasses and the remaining bagasse is utilized for feed or fuel. Unlike the juice of sugarcane, the sucrose in sweet sorghum juice cannot be crystallized to make table sugar as the ratio of sucrose to other sugars is too low. Sugarcane juice, by contrast, has an average of 94% sucrose, which makes crystallization feasible. Thus, providing sorghum plants with a sucrose purity greater than 94% would allow table sugar production from sweet sorghum juice.

### SUMMARY

The present disclosure features sorghum plants that have an increased total sugar content and increased sucrose purity at maturity. For example, the sorghum plants can have a sucrose purity of at least 90%, 91%, 92%, 93%, 94%, or 95% in the stalks at maturity. Surprisingly, plant sterility sequences that affect a developmental stage such as i) spikelet meristem identity, ii) establishment of floral meristem identity, or iii) floral organ initiation, development, or function can be used to increase the sucrose purity in sorghum plants.

In one aspect, a sorghum plant is featured that comprises an exogenous nucleic acid. The exogenous nucleic acid comprises a regulatory region operably linked to a plant sterility sequence, which affects a developmental stage selected from the group consisting of i) spikelet meristem identity, ii) establishment of floral meristem identity, and iii) floral organ initiation, development, or function. The stalk of the sorghum plant can has a sucrose purity that is higher at maturity than that of a corresponding control plant that lacks the exogenous nucleic acid. The stalk of the sorghum plant can have an increased total sugar content at maturity relative to that of the corresponding control plant. For example, the stalk can have a total sugar content that is increased by 12% or more (e.g., 25% or more, 30% or more, 12 to 25%, 40 to 60%) relative to a corresponding sorghum plant that lacks the exogenous nucleic acid. The stalk of such a sorghum plant can have a sucrose purity of at least 95% at maturity. The plant can also have reduced fertility. The stalk can have a total sugar content that is increased by more than 30%, more than 40%, more than 50%, or more than 60%, relative to a corresponding sorghum plant that lacks the exogenous nucleic acid. The sorghum plant can be an F₁ hybrid plant, or a male sterile plant, e.g., a plant that exhibits cytoplasmic male sterility (CMS).

In another aspect, a plurality of F₁ transgenic sorghum seeds are featured. The seeds comprise an exogenous nucleic acid comprising a promoter operably linked to a plant sterility sequence. The plant sterility sequence affects a developmental stage selected from the group consisting of i) spikelet meristem identity, ii) establishment of floral meristem identity, and iii) floral organ initiation, development, or function. F₁ sorghum plants grown from such F₁ seeds express the plant sterility sequence. The stalks of the sorghum plants can have a sucrose purity that is higher at maturity than that of a corresponding control plant that lacks the exogenous nucleic acid. The stalks of the sorghum plants can have an increased total sugar content at maturity relative to that of the corresponding control plant. For example, the stalks can have a total sugar content that is increased by 12% or more (e.g., 25% or more, 30% or more, 12 to 25%, 40 to 60%) relative to a corresponding sorghum plant that lacks the exogenous nucleic acid.

In another aspect, a method of making sorghum F₁ seeds is disclosed. The method comprises crossing a plurality of first sorghum plants and a plurality of second sorghum plants, in which the first or the second sorghum plants comprise an exogenous nucleic acid. The exogenous nucleic acid comprises a promoter operably linked to a plant sterility sequence, which affects a developmental stage selected from the group consisting of i) spikelet meristem identity, ii) establishment of floral meristem identity, and iii) floral organ initiation, development, or function. The first sorghum plants are male sterile and the second sorghum plants are male fertile and comprise a fertility restorer gene. F₁ seed is harvested from the first sorghum plants. Plants grown from the F₁ seed express the plant sterility sequence. The stalks of the sorghum plants can have a sucrose purity that is higher at maturity than that of a corresponding control plant that lacks the exogenous nucleic acid. The stalks of the sorghum plants can have an increased total sugar content at maturity relative to that of the corresponding control plant. For example, the stalks can have a total sugar content that is increased by 12% or more (e.g., 25% or more, 30% or more, 12 to 25%, 40 to 60%) relative to a corresponding sorghum plant that lacks the exogenous nucleic acid. The method can further comprise growing sorghum plants from the harvested seeds. In another aspect, a sweet sorghum plant made by the method is featured. The sweet sorghum plant has a sugar purity of 80% or greater at maturity.

In another aspect, a method of making sucrose crystals is disclosed. The method comprises extracting juice from one or more of the aforementioned plants and crystallizing sucrose from the juice.

In another aspect, this disclosure features F₁ transgenic sorghum seeds. Such seeds comprise a first exogenous nucleic acid comprising a transcription UAS and a first promoter. The UAS and first promoter are operably linked to a plant sterility sequence that sequence affects a developmental stage selected from the group consisting of i) spikelet meristem identity, ii) establishment of floral meristem identity, and iii) floral organ initiation, development, or function. Such seeds also comprise a second exogenous nucleic acid comprising a second promoter operably linked to a transcription factor that binds the UAS. Sorghum plants grown from the F₁ seeds express the plant sterility sequence, and the stalks have a higher sucrose purity at maturity relative to that of a corresponding control plant lacking the exogenous nucleic acid. In some embodiments, the F₁ plants exhibit reduced fertility.

In another aspect, this disclosure features a method of making a sorghum plant, comprising providing a first sorghum plant and a second sorghum plant. The first sorghum plant comprises a first exogenous nucleic acid. The first exogenous nucleic acid comprises a transcription UAS and a first promoter, operably linked to a plant sterility sequence that affects a developmental stage selected from the group consisting of i) spikelet meristem identity, ii) establishment of floral meristem identity, and iii) floral organ initiation, development, or function. The second sorghum plant comprises a second exogenous nucleic acid, comprised of a second promoter operably linked to a transcription factor that binds the UAS. A plurality of first sorghum plants are crossed to a plurality of second sorghum plants. In some cases, the first sorghum plants are male sterile and the second sorghum plants are male fertile and comprises a fertility restorer gene. In other cases, the second sorghum plants are male sterile and the first sorghum plants are male fertile and comprises a fertility restorer gene. F₁ seed is harvested from the male sterile sorghum plants. The F₁ sorghum plants grown from the F₁ seed express the plant sterility sequence. The stalks of the sorghum plants can have an increased total sugar content at maturity relative to that of the corresponding control plant. For example, the stalks can have a total sugar content that is increased by 12% or more (e.g., 25% or more, 30% or more, 12 to 25%, 40 to 60%) relative to a corresponding sorghum plant that lacks the exogenous nucleic acid. The stalks of such sorghum plants can have a sucrose purity of at least 95% at maturity. A sweet sorghum plant made by this method is also featured. Such a plant can have a sugar purity of 80% or greater at maturity. The stalks of the sorghum plants can have an increased total sugar content at maturity relative to that of the corresponding control plant. For example, the stalks can have a total sugar content that is increased by 12% or more (e.g., 25% or more, 30% or more, 12 to 25%, 40 to 60%) relative to a corresponding sorghum plant that lacks the exogenous nucleic acid. In some embodiments, the F₁ plants exhibit reduced fertility.

The sucrose purity obtained in the methods, seeds, or plants described herein can be at least 90%, 91%, 92%, 93%, 94%, 95%, 96%, or 97% at maturity.

The plant sterility sequence can be an antisense nucleic acid, a ribozyme, or a small interfering RNA. The plant sterility sequence can affects spikelet meristem identity and reduce expression of a polypeptide selected from the group consisting of FZP, GN1, DEP1, PAP2, SNB, LHS1, IFA1, IDS1, and RCN. The first promoter can be PD3796 (SEQ ID NO:20) or PD3800 (SEQ ID NO:21).

The transcription factor can be a chimeric transcription factor, e.g., have a binding domain selected from the group consisting of a Hap1, LexA, Lac Operon, ArgR, AraC, PDR3, GAL4, and LEU3 binding domain, and/or an activation domain selected from the group consisting of a VP16, C1 protein, ATMYB2, HAFL-1, ANT, ALM2, AvrXa10, Viviparous 1 (VP1), DOF, and RISBZ1 activation domain.

The plant sterility sequence can affect establishment of floral meristem identity and reduce expression of a polypeptide selected from the group consisting of APO1, LFY, CAL, DL, MADS6, AP1, and FUL. The first promoter can be CeresAnnt:8643934 (SEQ ID NO:22); CeresAnnt:8632648 (SEQ ID NO: 23); CeresAnnt:8681303 (SEQ ID NO: 24); or CeresAnnt:8642422 (SEQ ID NO: 25).

The plant sterility sequence can affect floral organ initiation, development, or function and reduce expression of a polypeptide selected from the group consisting of OsMADS2, AP3, MADS3, PI, SUPERWOMAN1, OsMADS8, OsMADS58, AP1, AG, and AP2. The plant sterility sequence can affect floral organ initiation, development, or function and reduce expression of SHP1, SHP2, ANT, and CRC. The first promoter can be CeresAnnt:8657974 (SEQ ID NO:26); CeresAnnt:8732691 (SEQ ID NO:27); CeresAnnt:8031970 (SEQ ID NO:28); or CeresAnnt:8669907 (SEQ ID NO:29).

The plant sterility sequence can reduce expression of a nucleic acid having at least 80% identity to a nucleotide sequence selected from the group consisting of SEQ ID NO: 1, 2, 3, 4, 5, and 6. The first promoter can be PD3796 (SEQ ID NO:20) or PD3800 (SEQ ID NO:21).

The plant sterility sequence can reduce expression of a nucleic acid having at least 80% identity to a nucleotide sequence set forth in SEQ ID NO: 7, 8, 9, 10, 11, and 12. The first promoter can be CeresAnnt:8643934 (SEQ ID NO:22); CeresAnnt:8632648 (SEQ ID NO: 23); CeresAnnt:8681303 (SEQ ID NO:24); and CeresAnnt:8642422 (SEQ ID NO:25).

The plant sterility sequence can reduce expression of a nucleic acid having at least 80% identity to a nucleotide sequence selected from the group consisting of SEQ ID NO:12, 13, 14, 15, 16, 17, 18, and 19. The first promoter can be CeresAnnt:8657974 (SEQ ID NO:26); CeresAnnt:8732691 (SEQ ID NO:27); CeresAnnt:8031970 (SEQ ID NO:28); and CeresAnnt:8669907 (SEQ ID NO:29).

This disclosure also features a method of growing sorghum, comprising growing any of the F₁ sorghum plants described herein and harvesting biomass from the sorghum plants. The biomass can comprise the stalks of such sorghum plants.

In another aspect, this disclosure features a process for making a biofuel (e.g., ethanol). The process can include harvesting biomass from sorghum plants (e.g., stalks of sorghum plants) grown from any of the F₁ seeds described herein to obtain harvested sorghum biomass; extracting sorghum juice from the harvested sorghum biomass to obtain extracted juice that includes sugar; using the sugar of the extracted juice in a fermentation reaction to produce a fermentation product that includes a biofuel; and isolating the biofuel from the fermentation product to obtain a composition comprising the biofuel. The composition can include anhydrous ethanol.

In another aspect, this disclosure features a process for making a biofuel (e.g., ethanol). The process can include harvesting biomass (e.g., stalks) from any of the sorghum plants described herein to obtain harvested sorghum biomass; extracting sorghum juice from the harvested sorghum biomass to obtain extracted juice that includes sugar; using the sugar of the extracted juice in a fermentation reaction to produce a fermentation product that includes a biofuel; and isolating the biofuel from the fermentation product to obtain a composition comprising the biofuel. The composition can include anhydrous ethanol.

This disclosure also features use of a plant sterility sequence in making a sorghum plant (e.g., sweet sorghum plant) with increased sugar and sucrose purity, wherein the plant sterility sequence reduces expression of a nucleic acid having at least 80% identity to a nucleotide sequence selected from the group consisting of SEQ ID NO: 1, 2, 3, 4, 5, 6, 7, 8, 9, 10, 12, 13, 14, 15, 16, 17, 18, and 19.

This disclosure also features use of a plant sterility sequence in making a sorghum plant (e.g., sweet sorghum plant) having stalks of with increased sucrose purity, wherein the plant sterility sequence affects a developmental stage selected from the group consisting of i) spikelet meristem identity, ii) establishment of floral meristem identity, and iii) floral organ initiation, development, or function. The plant sterility sequence can reduce expression of a nucleic acid having at least 80% identity to a nucleotide sequence selected from the group consisting of SEQ ID NO: 1, 2, 3, 4, 5, 6, 7, 8, 9, 10, 12, 13, 14, 15, 16, 17, 18, and 19.

In another aspect, this disclosure features use of a sorghum plant (e.g., sweet sorghum plant) in making ethanol, the plant including an exogenous nucleic acid comprising a regulatory region operably linked to plant sterility sequence that affects a developmental stage selected from the group consisting of i) spikelet meristem identity, ii) establishment of floral meristem identity, and iii) floral organ initiation, development, or function, wherein stalks of the plant have increased sucrose purity. The plant sterility sequence can reduce expression of a nucleic acid having at least 80% identity to a nucleotide sequence selected from the group consisting of SEQ ID NO: 1, 2, 3, 4, 5, 6, 7, 8, 9, 10, 12, 13, 14, 15, 16, 17, 18, and 19.

This disclosure also features use of a sorghum plant (e.g., sweet sorghum plant) in making crystalized sugar. The plants includes an exogenous nucleic acid comprising a regulatory region operably linked to plant sterility sequence, wherein the plant sterility sequence affects a developmental stage selected from the group consisting of i) spikelet meristem identity, ii) establishment of floral meristem identity, and iii) floral organ initiation, development, or function, wherein stalks of the plant have increased sugar content and increased sucrose purity. The plant sterility sequence can reduce expression of a nucleic acid having at least 80% identity to a nucleotide sequence selected from the group consisting of SEQ ID NO: 1, 2, 3, 4, 5, 6, 7, 8, 9, 10, 12, 13, 14, 15, 16, 17, 18, and 19.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. In some instances, features of the invention may consist essentially of that feature rather than comprise that feature. Section headings are provided merely for convenience. The word “comprising” in the claims may be replaced by “consisting essentially of” or with “consisting of,” according to standard practice in patent law.

Other features and advantages of the invention will be apparent from the following detailed description.

### DETAILED DESCRIPTION

This disclosure provides transgenic sorghum plants that have an increased sucrose purity in the stalks at maturity. The increased sucrose purity is based, at least in part, on developmentally appropriate expression of certain nucleic acid constructs that affect fertility in sorghum. In addition to having a high sucrose purity, sorghum plants described herein also can have one or more of the following properties: an increased brix value (an approximate amount of sugar as measured by, for example, a digital refractometer), an increased total sugar content, reduced susceptibility to ergot infection, or reduced lodging (e.g., from reduced weight of grain panicle). Furthermore, as discussed below, such sorghum plants have reduced fertility or are sterile, and can therefore be grown on a commercial scale with less concern about unwanted spread of transgenes present in such plants. Sterility in such sorghum plants can be scored in the field, which helps in assessing transgene effect and allows additional biocontainment actions, if desired, to be taken. Easy visual assessment also helps in breeding new varieties most likely to exhibit a desired sterility phenotype.

Transgenic sorghum plants described herein express a plant sterility sequence that affect a developmental stage such as establishment of spikelet meristem identity, establishment of floral meristem identity, or floral organ initiation, development, or function, resulting in a visible abnormality at the specified stage and in some cases, subsequent stages, which negatively influence normal reproductive development of the plant. See, for example, Thompson and Hake, Plant Phys., 149:38-45 (2009), for a review of the developmental stages in grass.

### I. DEFINITIONS

“Cell type-preferential promoter” or “tissue-preferential promoter” refers to a promoter that drives expression preferentially in a target cell type or tissue, respectively, but may also lead to some transcription in other cell types or tissues as well.

“Control plant” refers to a sorghum plant that does not contain the exogenous nucleic acid present in a transgenic plant of interest, but otherwise has the same or similar genetic background as such a transgenic plant. A suitable control plant can be a non-transgenic wild type plant, a non-transgenic segregant from a transformation experiment, or a transgenic plant that contains an exogenous nucleic acid other than the exogenous nucleic acid of interest.

“Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

“Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Expression” refers to the process of converting genetic information of a polynucleotide into RNA through transcription, which is catalyzed by an enzyme, RNA polymerase, and into protein, through translation of mRNA on ribosomes.

“Heterologous polypeptide” as used herein refers to a polypeptide that is not a naturally occurring polypeptide in a sorghum plant cell, e.g., a transgenic Sorghum bicolor plant transformed with and expressing the coding sequence for a nitrogen transporter polypeptide from a Zea mays plant.

“Nucleic acid” and “polynucleotide” are used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers.

“Operably linked” refers to the positioning of a regulatory region and a sequence to be transcribed in a nucleic acid so that the regulatory region is effective for regulating transcription or translation of the sequence. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site.

“Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

“Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation sequence (UAS). For example, a suitable enhancer is a cis-regulatory element (−212 to −154) from the upstream region of the octopine synthase (ocs) gene. From et al., Plant Cell, 1:977-984 (1989).

“Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

“Variety” refers to a population of sorghum plants that share constant characteristics which separate them from other plants of the same species. A variety is often, although not always, sold commercially. While possessing one or more distinctive traits, a variety is further characterized by a very small overall variation between individuals within that variety. A “line” as distinguished from a variety most often denotes a group of sweet sorghum plants used non-commercially, for example in plant research. A line typically displays little overall variation between individuals for one or more traits of interest, although there may be some variation between individuals for other traits.

### II. METHODS FOR MAKING SORGHUM WITH INCREASED TOTAL SUGAR AND SUCROSE PURITY

This document features methods for making F₁ sorghum seeds having an exogenous nucleic acid comprising a regulatory region operably linked to a plant sterility sequence. Stalks of F₁ sorghum plants grown from such F₁ seeds have a sucrose purity, i.e., the percentage of sucrose relative to the total extractable sugars content in juice extracted from mature stalks, of at least 80%. In some embodiments, the F₁ plants are grain-type sorghum plants. In other embodiments, the F₁ plants are sweet sorghum plants. For sweet sorghum plants, the sucrose purity at harvest is at least 80%, e.g., 85%, 90%, 91%, 92%, 93%, 94%, 95%, 96%, or even 97%. Surprisingly, stalks of such F₁ plants can also have a total sugar content, i.e., total of sucrose, glucose, and fructose, that is increased by 12% or more relative to corresponding F₁ sorghum plants that lack the exogenous nucleic acid. For example, the total sugar content can be increased by 15%, 20%, 25%, 12-25%, 30%, 35%, 40%, 45%, 50%, 55%, or 60%, relative to a corresponding sorghum plant that lacks the exogenous nucleic acid.

Sorghum plants are bred in most cases by self-pollination techniques. With the incorporation of male sterility (either genetic or cytoplasmic), however, cross pollination breeding techniques can be utilized. Thus, in one embodiment, methods described herein include crossing a plurality of first sorghum plants with a plurality of second sorghum plants. As explained in more detail below, one of the sets of sorghum plants contains an exogenous nucleic acid that comprises a regulatory region operably linked to a plant sterility sequence. The other set of sorghum plants can have one or more desirable characteristics that complement or are lacking in the set containing the plant sterility sequence.

In some embodiments, a two component system is used. For example, the first sorghum plants can contain at least one nucleic acid construct that comprises a) a transcription factor upstream activating sequence (UAS) and a first promoter that are operably linked to a plant sterility sequence. The second sorghum plants can contain a nucleic acid encoding a transcription factor that is effective for binding to the UAS.

Upon crossing of the two sorghum plants, seed development ensues. Expression of the transcription factor, either in F₁ seeds or F₁ plants, activates transcription of the plant sterility sequence, which in turn results in the F₁ plants being sterile. Transfer of these transgenes, or any other transgene(s) present in such plants, to other sorghum plants is minimized or eliminated because all, or substantially all, of the F₁ plants are sterile. Thus, unwanted spread of transgenes to other sorghum plants is effectively prevented.

Parent Plants

Suitable plants of Sorghum bicolor include inbred lines B.Tx635; B.Tx637; B.Tx627; B.Tx2752; B.Tx430, Wheatland, and C401. Also suitable are plants of Sorghum bicolor hybrids such as Pioneer Hi-Bred® 31 G65 (RR2) and DeKalb® DK-40Y. Also suitable are plants of Sorghum bicolor ssp. sudanense L. (Sorghum×drummondii). It is contemplated that plants of Sorghum×sudangrass hybrids (Sorghum bicolor×S. bicolor spp. sudanese) and Sorghum×almum hybrids may also be suitable. Also suitable are sweet sorghum varieties such as Umbrella, Della, Dale, Rio, Topper, M81, Sugar Drip, Wray, or N100.

A sorghum variety or line suitable for use as one of the parents in the methods described herein can be developed by plant breeding procedures generally described in, e.g., Allard, Principles of Plant Breeding, John Wiley & Sons, Inc. (1960); Simmonds, Principles of Crop Improvement, Longman Group Limited (1979); and, Jensen, Plant Breeding Methodology, John Wiley & Sons, Inc. (1988). Detailed breeding methodologies specifically applicable to sorghum take into account the necessity of reaching homozygosity for the transgene(s) that are to be present in the parent plants. See Section V below for further details on sorghum breeding.

Transgenic sorghum plants can be entered into a breeding program to introduce a different exogenous nucleic acid into the sorghum line or for further selection of other desirable traits, before using the plants as parents to make F₁ hybrids.

Transgene Inheritance

Sorghum plants that are to be used as parents in methods described herein are bred to exhibit homozygosity for the transgene(s) involved in conferring increased sucrose purity. Thus, for example, transgenic sorghum plants containing an exogenous nucleic acid (comprising a plant sterility sequence) are selected to be homozygous and exhibit simple Mendelian inheritance for the exogenous nucleic acid. As another example, transgenic sorghum plants containing a second exogenous nucleic acid (comprising a transcription factor coding sequence) are selected to be homozygous and exhibit simple Mendelian inheritance for the exogenous nucleic acid. As another example, transgenic sorghum plants containing a third exogenous nucleic acid (comprising a sequence of interest) are selected to be homozygous and exhibit simple Mendelian inheritance for the exogenous nucleic acid. In this regard, progeny testing via molecular analysis can be particularly useful during backcrossing to obtain a population that contains the exogenous nucleic acid. Polycross sib mating of the population followed by progeny testing to identify homozygous individuals can then yield the desired transgenic parent line.

Crossing Parent Plants

Sorghum plants are bred in most cases by self pollination techniques. With the incorporation of male sterility (either genetic or cytoplasmic), cross pollination breeding techniques can be utilized. Sorghum has a perfect flower with both male and female parts in the same flower located in the panicle. The flowers are usually in pairs on the panicle branches. Natural pollination occurs in sorghum when anthers (male flowers) open and pollen falls onto receptive stigma (female flowers). Because of the close proximity of male (anthers) and female (stigma) in the panicle, self pollination can be high. Cross pollination may occur when wind or convection currents move pollen from the anthers of one plant to receptive stigma on another plant. Cross pollination is enhanced with incorporation of male sterility, which renders male flowers nonviable without affecting the female flowers. Successful pollination in the case of male sterile flowers requires cross pollination.

The first and second sorghum parent plants are crossed by growing a plurality of the two types of plants in pollinating proximity. The two parent plants typically are planted in separate rows but can be randomly interplanted, and grown in a field under agronomic practices suitable for sorghum and known in the art. In either scheme, the ratio of first parent plants to second parent plants can vary from 1:10 to 10:1, e.g., the first parent:second parent ratio can be 9:1, 4:1, 1:1, 1:4, or 1:9. The choice of a suitable ratio can be made by one of ordinary skill based on factors such as pollen shed of the male parent and pollen receptivity of the female parent.

Collecting Seed

The F₁ seeds are collected at maturity, either by harvesting seeds from one of the parent plants (the female parent) or by harvesting seeds from both parent plants. Either technique of harvesting is encompassed by the methods described herein. F₁ hybrid seeds produced by the methods described herein can have reduced fertility, i.e., such seeds have a high germination percentage, but the resulting F₁ hybrid plants produce a decreased number of F₂ seeds. F₁ plants are considered to have reduced fertility when the average number of F₂ seed produced by such F₁ plants is about 5% to about 25% less than that from a corresponding non-transgenic plant. In some embodiments, the seeds are sterile, i.e., such seeds have a high germination percentage, but the resulting F₁ hybrid plants produce little or no F₂ seeds. F₁ plants are considered to be sterile when the average number of F₂ seed produced by such F₁ plants is less than 0.5 viable seeds per plant, e.g., less than 0.4, 0.3, 0.2, 0.1, 0.05, 0.01, or 0.005 fertile seeds per F₁ plant. F₁ plants are also considered to be sterile when the average number of F₂ seeds is so low as to be undetectable. Typically, a difference in the amount of a parameter relative to a control is considered statistically significant at p<0.05 with an appropriate parametric or non-parametric statistic, e.g., Chi-square test, Student's t-test, Mann-Whitney test, or F-test.

### III. NUCLEIC ACIDS

Plant Sterility Sequences.

Transgenic sorghum plants described herein contain an exogenous nucleic acid comprising a regulatory region operably linked to a plant sterility sequence such that gene expression is inhibited. As described herein, a plant sterility sequence affects establishment of spikelet meristem identity, establishment of floral meristem identity, or floral organ initiation, development, or function. A number of nucleic acid based methods, including antisense RNA, ribozyme directed RNA cleavage, post-transcriptional gene silencing (PTGS), e.g., RNA interference (RNAi), and transcriptional gene silencing (TGS) can be used to inhibit gene expression. Suitable polynucleotides include full-length nucleic acids encoding regulatory proteins or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid segment from a gene to be repressed is cloned and operably linked to a regulatory region and a transcription termination sequence so that the antisense strand of RNA is transcribed. The recombinant vector is then transformed into plants, as described below, and the antisense strand of RNA is produced. The nucleic acid segment need not be the entire sequence of the gene to be repressed, but typically will be substantially complementary to at least a portion of the sense strand of the gene to be repressed.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N. J. RNA endoribonucleases which have been described, such as the one that occurs naturally in Tetrahymena thermophile, can be useful. See, for example, U.S. Pat. Nos. 4,987,071 and 6,423,885.

PTGS, e.g., RNAi, can also be used to inhibit the expression of a gene. In some embodiments, a plant sterility sequence can be transcribed into a transcription product that inhibits expression of a polypeptide containing an AP2 domain, such as AP2, IDS 1 (Indeterminate Spikelet 1), SNB (Supernumerary bract, two AP2 domains), or IFA1 (indeterminate floral apex1). See, Chuck et al., Genes Dev., 12(8):1145-1154 (1998); Lee et al., Plant J., 49(1):64-78 (2006); and Laudencia-Chingcuanco and Hake, Development, 129(11):2629-38 (2002). IDS1, SNB, and IFA1 affect spikelet meristem identity while AP2 affects floral organ initiation, development, and function. SEQ ID NO:5 sets forth the nucleotide sequence of a Sorghum bicolor clone, identified herein as Ceres Annot ID No. 8645308 that is predicted to encode a SNB polypeptide containing two AP2 domains.

In some embodiments, a plant sterility sequence can be transcribed into a transcription product that inhibits expression of a polypeptide having a MADS box domain, e.g., LHS1 (Leafy hull sterile 1), FUL (fruitful), PAP2 (panicle phytomer 2), AP1 (Apetela1), AP3, MADS6 (also called MFO1, mosaic floral organ1) or CAL (Cauliflower, also known as AP1 or OsMADS14); a B-class MADS box protein such as PI (Pistillata), homologs of PI such as OsMADS2 (also known as GLO) or OsMADS4 (also known as GLO(2)); or a C-class MADS box protein such as AG (AGAMOUS), OsMADS3, OsMADS58 (homolog of AG), or SPW1 (Superwoman, also known as OsMADS16). See, e.g., Kobayashi et al., Plant Cell Physiol., 51(1): 47-57 (2010); Jeon et al., Plant Cell., 12(6):871-84 (2000); Alvarez-Buylla et al., J Exp Bot., 57(12):3099-107 (2006); Gu et al., Development, 125(8):1509-17 (1998); Yamaguchi et al., Plant Cell,18(1):15-28. (2006); Ohmori et al., Plant Cell, 21(10):3008-25 (2009), and Piwarzyk et al., Plant Physiol., 145(4):1495-505 (2007). PAP2 and LHS1 affect spikelet meristem identity. FUL, CAL, and AP1 affect floral meristem identity. CAL, AP1, AP3, PI, AG, OsMADS3, OsMADS4, OsMADS8, OsMADS58, and SPW1 affect floral organ initiation, development, or function. The MADS box domain is found in transcription factor proteins and can bind DNA. Proteins belonging to the MADS family function as dimers, each subunit of which contributes an amphipathic alpha helix to form the anti-parallel coiled-coil DNA-binding element. The MADS-box domain is commonly associated with a K-box region, which is predicted to have a coiled-coil structure and play a role in multimer formation. SEQ ID NO:4 sets forth the nucleotide sequence of a Sorghum bicolor clone, identified herein as Ceres Annot ID No. 8632646 that is predicted to encode a PAP2 polypeptide containing a MADS box domain. SEQ ID NO:6 sets forth the nucleotide sequence of a Sorghum bicolor clone, identified herein as Ceres Annot ID no. 8642422 that is predicted to encode a LHS1 polypeptide containing a MADS box domain. SEQ ID NO:9 sets forth the nucleotide sequence of a Sorghum bicolor clone, identified herein as Ceres Annot ID No. 8632648 that is predicted to encode a CAL polypeptide containing a MADS box domain. SEQ ID NO:11 sets forth the nucleotide sequence of a Sorghum bicolor clone, identified herein as Ceres Annot ID No. 8681303 that is predicted to encode a MADS6 polypeptide containing a MADS box domain. SEQ ID NO:12 sets forth the nucleotide sequence of a Sorghum bicolor clone, identified herein as Ceres Annot ID no. 8643934 that is predicted to encode an AP1 polypeptide containing a MADS box domain. SEQ ID NO:13 sets forth the nucleotide sequence of a Sorghum bicolor clone, identified herein as Ceres Annot ID No. 8669907 that is predicted to encode a PI polypeptide containing a MADS box domain. SEQ ID NO:14 sets forth the nucleotide sequence of a Sorghum bicolor clone, identified herein as Ceres Annot ID No. 8744657 that is predicted to encode an AP3 polypeptide containing a MADS box domain. SEQ ID NO:15 sets forth the nucleotide sequence of a Sorghum bicolor clone, identified herein as Ceres Annot ID No. 8657974 that is predicted to encode an MADS3 polypeptide containing a MADS box domain. SEQ ID NO:16 sets forth the nucleotide sequence of a Sorghum bicolor clone, identified herein as Ceres Annot ID No. 8732691 that is predicted to encode an MADS4 polypeptide containing a MADS box domain. SEQ ID NO:17 sets forth the nucleotide sequence of a Sorghum bicolor clone, identified herein as Ceres Annot ID No. 8031970 that is predicted to encode an SPW1 polypeptide containing a MADS box domain. SEQ ID NO:19 sets forth the nucleotide sequence of a Sorghum bicolor clone, identified herein as Ceres Annot ID No. 8725895 that is predicted to encode a MADS58 polypeptide containing a MADS box domain.

In some embodiments, a plant sterility sequence can be transcribed into a transcription product that inhibits expression of a polypeptide having an F box domain, such as APO1 (aberrant panicle organization 1). See, e.g., Ikeda et al., Plant J., 51(6):1030-1040 (2007). APO1 affect spikelet meristem identity. An F box domain typically is about 50 amino acids long, and is usually found in the N-terminal half of a protein. An F-box domain can include leucine rich repeats and the WD repeat. The F-box domain helps mediate protein-protein interactions in a variety of contexts, including polyubiquitination, transcription elongation, centromere binding and translational repression. SEQ ID NO:7 sets forth the nucleotide sequence of a Sorghum bicolor clone, identified herein as Ceres Annot ID No. 8743976 that is predicted to encode a polypeptide containing an F box domain.

In some embodiments, a plant sterility sequence can be transcribed into a transcription product that inhibits expression of a polypeptide having an ERF (ethylene-responsive element-binding factor) domain, such as branched silkless 1) and FZP (Frizzle panicle, homolog of BD1). See, e.g., Komatsu et al., supra (2003). BD1 and FZP affect floral meristem identity. An ERF domain is found in transcription factors and can specifically bind to the GCC box AGCCGCC, which is involved in the ethylene-responsive transcription of genes. See, e.g., Komatsu et al., Development, 130:3841-3850 (2003). SEQ ID NO:1 sets forth the nucleotide sequence of a Sorghum bicolor clone, identified herein as Ceres Annot ID No. 8657227 that is predicted to encode an FZP polypeptide containing an ERF domain.

In some embodiments, a plant sterility sequence can be transcribed into a transcription product that inhibits expression of a polypeptide having an N-terminal proline rich domain and a conserved C-terminal domain, such as LFY (Leafy). See, e.g., Rao et al., Proc. Natl. Acad. Sci., 105(9):3646-3651 (2008). LY affects establishment of spikelet meristem identity and floral meristem identity. SEQ ID NO:8 sets forth the nucleotide sequence of a Panicum virgatum clone, identified herein as Ceres Clone Id No. 8702677 that is predicted to encode an N-terminal proline rich domain and a conserved C-terminal domain.

In some embodiments, a plant sterility sequence can be transcribed into a transcription product that inhibits expression of a polypeptide having a cytokinin/dehydrogenase activity, such as GN1 (OsCKX2), an enzyme that degrades the phytohormone cytokinin. See, e.g., Ashikari et al., Science, 309(5735):741-5 (2005). GN1 affects establishment of spikelet meristem identity. SEQ ID NO:2 sets forth the nucleotide sequence of a Sorghum bicolor clone, identified herein as Ceres Annot ID No. 86580247 that is predicted to encode a GN1 polypeptide.

In some embodiments, a plant sterility sequence can be transcribed into a transcription product that inhibits expression of a transcription factor containing a zinc-finger and helix-loop-helix domain (referred to as a YABBY domain), such as DL (DROOPING LEAF, also known as Superman1). DL is a member of the YABBY gene family and is closely related to the CRABS CLAW (CRC) gene of Arabidopsis thaliana. See, e.g., Yamaguchi et al., Plant Cell. 16(2): 500-509 (2004). DL affects establishment of floral meristem identity. SEQ ID NO:10 sets forth the nucleotide sequence of a Sorghum bicolor clone, identified herein as Ceres Annot ID No. 8642423 that is predicted to encode a DL polypeptide.

In some embodiments, a plant sterility sequence can be transcribed into a transcription product that inhibits expression of a gene that regulates fertility, such as Dense and Erect Panicle1 (DEP1). DEP1 encodes a protein containing the phosphatidylethanolamine-binding protein (PEBP) domain. See, e.g., Wang, Curr Opin Plant Biol. 14(1):94-9. Epub 2010 Dec. 6 (2011). DEP1 affects establishment of spikelet meristem identity. SEQ ID NO:3 sets forth the nucleotide sequence of a Sorghum bicolor clone, identified herein as Ceres Annot ID No. 865436 that is predicted to encode a DEP1 polypeptide.

For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence of the polypeptide of interest, or a fragment thereof, and that is from about 10 nucleotides to about 2,500 nucleotides in length. For example, the length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand, or a fragment thereof, of the coding sequence of the polypeptide of interest, and can have a length that is shorter, the same as, or longer than the corresponding length of the sense sequence. In some cases, one strand of the stem portion of a double stranded RNA comprises a sequence that is similar or identical to the 3′ or 5′ untranslated region, or a fragment thereof, of the mRNA encoding the polypeptide of interest, and the other strand of the stem portion of the double stranded RNA comprises a sequence that is similar or identical to the sequence that is complementary to the 3′ or 5′ untranslated region, respectively, of the mRNA encoding the polypeptide of interest. In other embodiments, one strand of the stem portion of a double stranded RNA comprises a sequence that is similar or identical to the sequence of an intron, or a fragment thereof, in the pre-mRNA encoding the polypeptide of interest, and the other strand of the stem portion comprises a sequence that is similar or identical to the sequence that is complementary to the sequence of the intron, or a fragment thereof, in the pre-mRNA.

The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron, or a fragment thereof. A double stranded RNA can have zero, one, two, three, four, five, six, seven, eight, nine, ten, or more stem-loop structures.

A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing a regulatory region operably linked to a nucleic acid in sense orientation can also be used to inhibit the expression of a gene. The transcription product can be similar or identical to the sense coding sequence, or a fragment thereof, of a polypeptide of interest. The transcription product can also be unpolyadenylated, lack a 5′ cap structure, or contain an unspliceable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the full-length sequence, or a fragment thereof, of an mRNA, the 3′ or 5′ untranslated region of an mRNA, or an intron in a pre-mRNA encoding a polypeptide of interest. In some embodiments, the sense or antisense sequence is identical or complementary to a sequence of the regulatory region, or a fragment thereof, that drives transcription of the gene encoding a polypeptide of interest. In each case, the sense sequence is the sequence that is complementary to the antisense sequence.

The sense and antisense sequences can be any length greater than about 12 nucleotides (e.g., 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence encoding a polypeptide described herein. The sense sequence complementary to the antisense sequence can be a sequence present within the mRNA of a polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or P-DNA such that the left and right T-DNA border sequences, or the left and right border-like sequences of the P-DNA, flank or are on either side of the nucleic acid. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some embodiments, a nucleic acid as described above is designed to inhibit expression of more than one gene in a plant. Such a nucleic acid has fragment(s) from a first gene to be inhibited as well as fragment(s) from a second, third or even fourth gene to be inhibited. For example, a construct can be used to target Shatterproof1 (SHP1), SHP2, aintegumenta (ANT) and crabs claw (CRC). See, for example, Colombo et al., Dev Biol. 337(2):294-302 (2010).

In some embodiments, a plant sterility sequence used to inhibit gene expression has at least 80% identity (e.g., 85%, 90%, 95%, 98%, 99%, or 100% identity) to the target sequence. “Percent sequence identity” refers to the degree of sequence identity between any given reference sequence, e.g., SEQ ID NO:1, and a candidate plant sterility sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw). To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some embodiments, a plant sterility sequences reduces expression of a functional homolog of a target. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a plant sterility polypeptide, or by combining domains from the coding sequences for different naturally-occurring plant sterility polypeptides (“domain swapping”). The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of plant sterility polypeptides. Sequence analysis can involve BLAST, Reciprocal BLAST, or PSI-BLAST analysis of nonredundant databases using a plant sterility polypeptide amino acid sequence as the reference sequence. Amino acid sequence is, in some instances, deduced from the nucleotide sequence. Those polypeptides in the database that have greater than 40% sequence identity are candidates for further evaluation for suitability as a plant sterility polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in plant sterility polypeptides, e.g., conserved functional domains.

Conserved regions can be identified by locating a region within the primary amino acid sequence of a plant sterility polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

The identification of conserved regions in a plant sterility polypeptide facilitates production of variants of plant sterility polypeptides. Variants of plant sterility polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions.

In some embodiments, a target sequence encodes a polypeptide that fits a Hidden Markov Model. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, --consistency REPS of 2; -ir, --iterative-refinement REPS of 100; -pre, --pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer.wustl.edu; and fr.com/hmmer232/. Hmmbuild outputs the model as a text file.

The HMM for a group of functional homologs can be used to determine the likelihood that a candidate plant sterility polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

Transcription Factors.

In some embodiments, a two components system is used to control expression of the plant sterility sequence. With the two component system, F₁ transgenic sorghum plants contain an exogenous nucleic acid encoding a transcription factor that activates transcription of the plant sterility sequence linked to an upstream activating sequence. Transcription factors typically have discrete DNA binding and transcription activation domains. The DNA binding domain(s) and transcription activation domain(s) of transcription factors can be synthetic or can be derived from different sources (i.e., be chimeric transcription factors). It is known that domains from different naturally occurring transcription factors can be combined in a single polypeptide and that expression of such a chimeric transcription factor in plants can activate transcription. In some embodiments, a chimeric transcription factor has a DNA binding domain derived from the yeast Ga14 gene and a transcription activation domain derived from the VP16 gene of herpes simplex virus. In other embodiments, a chimeric transcription factor has a DNA binding domain derived from a yeast HAP 1 gene and the transcription activation domain derived from VP16. See, e.g., WO 97/30164.

A list of DNA binding domains from various transcription factors is shown in Table 1, along with their respective upstream activation sequences. These domains are suitable for use in a chimeric transcription factor in sorghum. DNA-binding domains on this list have been expressed in transgenic plants as components of chimeric transcription factors. It is contemplated that the DNA binding domain from a S. cerevisiae LEU3 transcription factor and its associated UAS (CCG-N4-CGG) and the DNA binding domain from a S. cerevisiae PDR3 transcription factor and its associated UAS (CCGCGG) will also be suitable. See, Hellauer et al., Mol. Cell Biol. (1996).

TABLE 1
Binding Domains

| Transcription | Source | | |
|---|---|---|---|
| Factor Name | Organism | UAS | Reference |
| HAP1 | S. | agcaCGGacttatCGGtcgg (SEQ | WO 97/30164 |
| | cerevisiae | ID NO: 30) | |
| | | GcagCGGtattaaCGGgattac | |
| | | (SEQ ID NO: 31) | |
| | | 5′Nnnn CGG nnntan CGG | SEQ ID NO: 37 |
| | | NNNta | |
| LexA | E. coli | TACTG(TA)5CAGTA (SEQ ID | U.S. Pat. No. 6,399,857; U.S. |
| | | NO: 32) | Pat. No. 6,946,586; Wade et |
| | | | al, Genes & Dev. |
| | | | 19: 2619-2630, 2005 |
| Lac Operon | E. coli | AATTGTGAGCGCTCACAATT | Moore et al. PNAS Jan |
| | | (SEQ ID NO: 33) | 6; 95(1): 376-81 (1998); |
| | | | U.S. Pat. No. 6,172,279 |
| ArgR | E. coli | wNTGAAT-w4-ATTCANw | Werner K Maas, |
| | | (SEQ ID NO: 34) | Microbiol Review, |
| | | | 1994 Vol 58, pp. 631- |
| | | | 640 |
| AraC | E. coli | TATGGATAAAAATGCTA | Bustos and Schleif, |
| | | (SEQ ID NO: 35) | 1993 |
| Synthetic Zn | N/A | N/A | U.S. Pat. No. 7,273,923; |
| proteins | | | U.S. Pat. No. 7,262,054 |
| Gal4 | S. | SEQ ID NO: 36 | See SEQ ID NO: 38 for |
| | cerevisiae | | GAL4 DNA binding |
| | | | domain |

A list of transcription activation domains from various transcription factors is shown in Table 2, along with the amino acid residues where the domain is located in the protein. These domains are suitable for use in a chimeric transcription factor in sorghum. Most of the activation domains on this list have been shown to be functional in heterologous plant systems.

TABLE 2
Activation Domains

| | | Domain Location | |
|---|---|---|---|
| Transcription | | (Amino Acid | |
| Factor Name | Organism | Residue Nos.) | Reference |
| C1 protein | Maize | 173-273 | Goff SA et al., Gene & Dev. (1991). |
| | | | Van Eenenaam et al. Metab Eng. |
| | | | (2004) |
| ATMYB2 | Arabidopsis | 146-269 | Urao et al., Plant J. (1996) |
| HAFL-1 | Wheat | 214-273 | Okanami et al. Genes to Cells (1996) |
| ANT | Arabidopsis | 221-274 | Krizek & Sulli, Planta (2006) |
| ALM2 | Arabidopsis | 203-256 | Anderson & Hanson, BMC Plant Biol. |
| | | | (2005) |
| AvrXa10 | Xanthomonas oryzae | 133-274 | Zhu et al. Plant Cell 1999 |
| | pv. oryzae | | |
| Viviparous 1 (VP1) | Maize | 134-213 | McCarty et al. Cell (1991) |
| DOF | Maize | 1-163 | Yanagisawaa & Sheen Plant Cell |
| | | | (1998) |
| RISBZ1 | Rice | 1060-1102 | Onodera et al., J. Biol. Chem. (2001) |
| VP16 | Herpes simplex | 411-490 | Greaves and O'Hare, J. Virol., |
| | | | 63: 1641-1650 (1989) |

Regulatory Regions

The choice of regulatory regions to be included in a recombinant construct depends upon several factors, including, but not limited to, efficiency, selectability, inducibility, desired expression level, and cell- or tissue-preferential expression. For example, to affect the establishment of spikelet meristem identity, a promoter such as PD3796 (SEQ ID NO:20) or PD3800 (SEQ ID NO:21), or functional fragments thereof, can be used in a nucleic acid construct. To affect the establishment of floral meristem identity, a promoter such as CeresAnnt:8643934 (SEQ ID NO:22), CeresAnnt:8632648 (SEQ ID NO:23), CeresAnnt:8681303 (SEQ ID NO:24), or CeresAnnt:8642422 (SEQ ID NO:25), or functional fragments thereof, can be used in a nucleic acid construct. To affect floral organ initiation, development, or function, a promoter such as CeresAnnt:8657974 (SEQ ID NO:26), CeresAnnt:8732691 (SEQ ID NO:27), CeresAnnt:8031970 (SEQ ID NO:28), or CeresAnnt:8669907 (SEQ ID NO:29), or functional fragments thereof, can be used in a nucleic acid construct. It is a routine matter for one of skill in the art to position regulatory regions relative to the coding sequence and to identify functional fragments of regulatory regions.

For example, methods for identifying and characterizing regulatory regions in plant genomic DNA, include those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996). In one embodiment, the ability of regulatory regions of varying lengths to direct expression of an operably linked nucleic acid can be assayed by operably linking varying lengths of a regulatory region to a reporter nucleic acid and transiently or stably transforming a cell, e.g., a plant cell, with such a construct. Suitable reporter nucleic acids include β-glucuronidase (GUS), green fluorescent protein (GFP), yellow fluorescent protein (YFP), and luciferase (LUC). Expression of the gene product encoded by the reporter nucleic acid can be monitored in such transformed cells using standard techniques.

Examples of various classes of regulatory regions are described below. Some of the regulatory regions indicated below as well as additional regulatory regions are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; and PCT/US07/62762.

For example, the sequences of regulatory regions p326, PD2995, PD3141, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PR0924 is set forth in the sequence listing of PCT/US07/62762; the sequences of regulatory regions p530c10, pOsFIE2-2, pOsMEA, pOsYp102, and pOsYp285 are set forth in the sequence listing of PCT/US06/038236; the sequence of PD2995 is set forth in the sequence listing of PCT/US09/32485; and the sequence of PD3141 promoter is set forth in the sequence listing of PCT/US09/32485.

It will be appreciated that a regulatory region may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326, PD2995, YP0144, YP0190, p13879, YP0050, p32449, 21876, YP0158, YP0214, YP0380, PT0848, and PT0633 promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380 and PT0585.

Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380, PT0848, YP0381, YP0337, PT0633, YP0374, PT0710, YP0356, YP0385, YP0396, YP0388, YP0384, PT0688, YP0286, YP0377, PD1367, and PD0901. Examples of nitrogen-inducible promoters include PT0863, PT0829, PT0665, and PT0886. Examples of shade-inducible promoters include PR0924 and PT0678. An example of a promoter induced by salt is rd29A (Kasuga et al. (1999) Nature Biotech 17: 287-291).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, shoot-preferential, parenchyma cell-preferential, and senescence-preferential promoters. In some embodiments, a promoter may preferentially drive expression in reproductive tissues (e.g., PO2916 promoter, SEQ ID NO:31 in 61/364,903). Promoters designated YP0086, YP0188, YP0263, PT0758, PT0743, PT0829, YP0119, and YP0096, as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a heat and/or drought-tolerance polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Nucleic Acid Expression.

For expression of a plant sterility sequence, a suitable nucleic acid encoding a gene product is operably linked to a regulatory region (e.g., a promoter). In some embodiments, a suitable nucleic acid encoding a gene product is operably linked to a promoter and a UAS for a transcription factor. For expression of a transcription factor, a transcription factor coding sequence is operably linked to a promoter. As used herein, the term “operably linked” refers to positioning of a regulatory region in a nucleic acid so as to allow or facilitate transcription of the nucleic acid to which it is linked. For example, a recognition site for a transcription factor is positioned with respect to a promoter so that upon binding of the transcription factor to the recognition site, the level of transcription from the promoter is increased. The position of the recognition site relative to the promoter can be varied for different transcription factors, in order to achieve the desired increase in the level of transcription. Selection and positioning of promoter and transcription factor recognition site is affected by several factors, including, but not limited to, desired expression level, cell or tissue specificity, and inducibility.

A nucleic acid for use in the invention may be obtained by, for example, DNA synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach, C. & Dveksler, G., Eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are available by which site-specific nucleotide sequence modifications can be introduced into a template nucleic acid.

Nucleic acids for use in the invention may be detected by techniques such as ethidium bromide staining of agarose gels, Southern or Northern blot hybridization, PCR or in situ hybridizations. Hybridization typically involves Southern or Northern blotting. See e.g., Sambrook et al., 1989, Molecular Cloning: A Laboratory Manual, 2^{nd} Edition, Cold Spring Harbor Press, Plainview, N.Y., sections 9.37-9.52. Probes should hybridize under high stringency conditions to a nucleic acid or the complement thereof. High stringency conditions can include the use of low ionic strength and high temperature washes, for example 0.015 M NaCl/0.0015 M sodium citrate (0.1×SSC), 0.1% sodium dodecyl sulfate (SDS) at 65° C. In addition, denaturing agents, such as formamide, can be employed during high stringency hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Herbicide Tolerance

In addition to the other exogenous nucleic acids described herein, sorghum plants can contain a transgene that confers herbicide resistance. Herbicide resistance is also sometimes referred herein to as herbicide tolerance. Expression of a herbicide resistance transgene is regulated independently of plant sterility sequences in plants, i.e., is not regulated by transcription factors encoded by exogenous nucleic acids. Polypeptides conferring resistance to a herbicide that inhibits the growing point or meristem, such as an imidazolinone or a sulfonylurea can be suitable. Exemplary polypeptides in this category code for mutant ALS and AHAS enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS) resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase(ACCase).

Polypeptides for resistance to glyphosate (sold under the trade name Roundup®) are also suitable. See, for example, U.S. Pat. No. 4,940,835 and U.S. Pat. No. 4,769,061. U.S. Pat. No. 5,554,798 discloses transgenic glyphosate resistant maize plants, in which resistance is conferred by an altered 5-enolpyruvyl-3-phosphoshikimate (EPSP) synthase. Such polypeptides can confer resistance to glyphosate herbicidal compositions, including without limitation glyphosate salts such as the trimethylsulphonium salt, the isopropylamine salt, the sodium salt, the potassium salt and the ammonium salt. See, e.g., U.S. Pat. Nos. 6,451,735 and 6,451,732.

Polypeptides for resistance to phosphono compounds such as glufosinate ammonium or phosphinothricin, and pyridinoxy or phenoxy propionic acids and cyclohexones are also suitable. See European application No. 0 242 246. See also, U.S. Pat. Nos. 5,879,903, 5,276,268 and 5,561,236.

Other herbicides include those that inhibit photosynthesis, such as a triazine and a benzonitrile (nitrilase). See U.S. Pat. No. 4,810,648. Other herbicides include 2,2-dichloropropionic acid, sethoxydim, haloxyfop, imidazolinone herbicides, sulfonylurea herbicides, triazolopyrimidine herbicides, s-triazine herbicides and bromoxynil. Also suitable are herbicides such as isoxazoles that inhibit hydroxyphenylpyruvate dioxygenases. Also suitable are herbicides that confer resistance to a protox enzyme. See, e.g., U.S. Patent Application No. 20010016956, and U.S. Pat. No. 6,084,155.

Transformation

Techniques for introducing exogenous nucleic acids into sorghum plants include, without limitation, Agrobacterium-mediated transformation and particle gun transformation. See, e.g., PCT/US2011/022738 and Tadesse, et al., Plant Cell Tissue Organ Cult 75, 1-18 (2003), respectively. Agrobacterium-mediated transformation is particularly useful. If a cell or tissue culture is used as the recipient tissue for transformation, plants can be regenerated from transformed cultures by techniques known to those skilled in the art.

### IV. SEQUENCES OF INTEREST

Sorghum cells and plants described herein can also have an exogenous nucleic acid that comprises a sequence of interest, which is preselected for its beneficial effect upon a trait of commercial value. An exogenous nucleic acid comprising a sequence of interest is operably linked to a regulatory region for transformation into sorghum plants, and plants are selected whose expression of the sequence of interest achieves a desired amount and/or specificity of expression. A suitable regulatory region is chosen as described herein. In most cases, expression of a sequence of interest is regulated independently of plant sterility sequences in plants, i.e., is not regulated by exogenous nucleic acids encoding transcription factors as described herein. It will be appreciated, however, that in some embodiments expression of a sequence of interest is regulated by transcription factors that regulate plant sterility sequences as described herein.

A sequence of interest can encode a polypeptide or can regulate the expression of a polypeptide. A sequence of interest that encodes a polypeptide can encode a plant polypeptide, a non-plant polypeptide such as a mammalian polypeptide, a modified polypeptide, a synthetic polypeptide, or a portion of a polypeptide. In some embodiments, a sequence of interest is transcribed into an antisense or interfering RNA molecule.

More than one sequence of interest can be present in a plant, e.g., two, three, four, five, six, seven, eight, nine, or ten sequences of interest can be present in a plant. Each sequence of interest can be present on the same nucleic acid construct or can be present on separate nucleic acid constructs. The regulatory region operably linked to each sequence of interest can be the same or can be different.

Lignin Biosynthesis Sequences

In certain cases, a sequence of interest can be an endogenous or exogenous sequence associated with lignin biosynthesis. For example, transgenic sorghum containing a recombinant nucleic acid encoding a regulatory protein can be effective for modulating the amount and/or rate of lignin biosynthesis. Such effects on lignin biosynthesis typically occur via modulation of transcription of one or more endogenous or exogenous sequences of interest operably linked to an associated regulatory region, e.g., endogenous genes involved in lignin biosynthesis, such as native enzymes or regulatory proteins in lignin biosynthesis pathways, or exogenous sequences involved in lignin biosynthesis pathways introduced via a recombinant nucleic acid construct into a plant cell.

In some embodiments, the coding sequence can encode a polypeptide involved in lignin biosynthesis, e.g., an enzyme or a regulatory protein (such as a transcription factor) involved in lignin biosynthesis described herein. Other components that may be present in a sequence of interest include introns, enhancers, upstream activation regions, and inducible elements.

A suitable sequence of interest can encode an enzyme involved in lignin biosynthesis, such as 4-(hydroxy)cinnamoyl CoA ligase (4CL; EC 6.2.1.12), p-coumarate 3-hydroxylase (C3H), cinnamate 4-hydroxylase (C4H; EC 1.14.13.11), cinnamyl alcohol dehydrogenase (CAD; EC 1.1.1.195), caffeoyl CoA O-methyltransferase (CCoAOMT; EC 2.1.1.104), cinnamoyl CoA reductase (CCR; EC 1.2.1.44), caffeic acid/5-hydroxyferulic acid O-methyltransferase (COMT; EC 2.1.1.68), hydroxycinnamoyl CoA:quinate hydroxycinnamoyltransferase (CQT; EC 2.3.1.99), hydroxycinnamoyl CoA:shikimate hydroxycinnamoyltransferase (CST; EC 2.3.1.133), ferulate 5-hydroxylase (F5H), phenylalanine ammonia-lyase (PAL; EC 4.3.1.5), p-coumaryl CoA 3-hydroxylase (pCCoA3H), or sinapyl alcohol dehydrogenase (SAD).

In some embodiments, a suitable sequence of interest can encode an enzyme involved in polymerization of lignin monomers to form lignin, such as a peroxidase (EC 1.11.1.x) or a laccase (EC 1.10.3.2) enzyme. In some cases, a suitable sequence of interest can encode an enzyme involved in glycosylation of lignin monomers, such as a coniferyl-alcohol glucosyltransferase (EC 2.4.1.111) enzyme, or an enzyme involved in regenerating a monolignol from a monolignol glucoside, such as a coniferin β-glucosidase (EC 3.2.1.126) enzyme. As mentioned above, such a suitable sequence of interest can be transcribed into an anti-sense or interfering RNA molecule.

Phenylpropanoid Sequences of Interest

In some embodiments, a sequence of interest can encode an enzyme involved in flavonoid biosynthesis, such as naringenin-chalcone synthase (EC 2.3.1.74), polyketide reductase, chalcone isomerase (EC 5.5.1.6), flavanone 4-reductase (EC 1.1.1.234), dihydrokaempferol 4-reductase (EC 1.1.1.219), flavone synthase (EC 1.14.11.22), flavone 7-O-beta-glucosyltransferase (EC 2.4.1.81), flavone apiosyltransferase (EC 2.4.2.25), isoflavone-7-O-beta-glucoside 6″-O-malonyltransferase (EC 2.3.1.115), apigenin 4′-O-methyltransferase (EC 2.1.1.75), flavonoid 3′-monooxygenase (EC 1.14.13.21), luteolin O-methyltransferase (EC 2.1.1.42), flavonoid 3′,5′-hydroxylase (EC 1.14.13.88), 4′-methoxyisoflavone 2′-hydroxylase (EC 1.14.13.53), isoflavone 4′-O-methyltransferase (EC 2.1.1.46), flavanone 3-dioxygenase (EC 1.14.11.9), leucocyanidin oxygenase (EC 1.14.11.19), flavonol synthase (EC 1.14.11.23), 2′-hydroxyisoflavone reductase (EC 1.3.1.45), leucoanthocyanidin reductase (EC 1.17.1.3), anthocyanidin reductase (EC 1.3.1.77), flavonol 3-O-glucosyltransferase (EC 2.4.1.91), quercetin 3-O-methyltransferase (EC 2.1.1.76), anthocyanidin 3-O-glucosyltransferase (EC 2.4.1.115), flavonol-3-O-glucoside L-rhamnosyltransferase (EC 2.4.1.159), UDP-glucose:anthocyanin 5-O-glucosyltransferase (2.4.1.-), or anthocyanin acyltransferase (2.3.1.-).

In some embodiments, a sequence of interest can encode an enzyme involved in stilbene synthesis such as trihydroxystilbene synthase (EC 2.3.1.95) or an oxidoreductase (EC 1.14.-.-).

In some embodiments, a sequence of interest can encode an enzyme involved in coumarin synthesis such as trans-cinnamate 2-monooxygenase (EC 1.14.13.14), 2-coumarate O-beta-glucosyltransferase (EC 2.4.1.114), a cis-trans-isomerase (EC 5.2.1.-), or a beta-glucosidase (EC 3.2.1.21).

Biomass-Modulating Sequences of Interest

Sequences of interest include those encoding a biomass-modulating polypeptide that contains at least one domain indicative of biomass-modulating polypeptides.

For example, a biomass-modulating polypeptide can contain a polyprenyl synthetase domain, which is predicted to be characteristic of a polyprenyl synthetase enzyme. A polyprenyl synthetase is a variety of isoprenoid compound which can be synthesized by various organisms. For example, in eukaryotes the isoprenoid biosynthetic pathway can be responsible for the synthesis of a variety of end products including cholesterol, dolichol, ubiquinone or coenzyme Q. In bacteria, this pathway can lead to the synthesis of isopentenyl tRNA, isoprenoid quinones, and sugar carrier lipids. Among the enzymes that can participate in that pathway, are a number of polyprenyl synthetase enzymes which catalyze a 1′4-condensation between 5 carbon isoprene units. All the above enzymes typically share some regions of sequence similarity. Two of these regions are typically rich in aspartic-acid residues and could be involved in the catalytic mechanism and/or the binding of the substrates.

A biomass-modulating polypeptide can contain a multiprotein bridging factor 1 domain. This domain forms a heterodimer with MBF2. It can make direct contact with the TATA-box binding protein (TBP) and can interact with Ftz-F1, stabilising the Ftz-F1-DNA complex. It can also be found in the endothelial differentiation-related factor (EDF-1). The domain can be found in a wide range of eukaryotic proteins including metazoans, fungi and plants. A helix-turn-helix motif (PF01381) is typically found to its C-terminus.

A biomass-modulating polypeptide can contain a Helix-turn-helix 3 domain. DNA binding helix-turn helix proteins include bacterial plasmid copy control protein, bacterial methylases, various bacteriophage transcription control proteins and a vegetative specific protein from Dictyostelium discoideum (Slime mold).

A biomass-modulating polypeptide can contain a plant neutral invertase domain, such as Bac_rhamnosid, GDE_C, Invertase_neut, and Trehalase.

A biomass-modulating polypeptide can contain a sedlin, N-terminal domain. Sedlin is a 140 amino-acid protein with a role in endoplasmic reticulum-to-Golgi transport.

A biomass-modulating polypeptide can contain a G-box binding protein MFMR domain. The domain is typically found to the N-terminus of the PF00170 transcription factor domain. It is typically between 150 and 200 amino acids in length. The N-terminal half is typically rather rich in proline residues and has been termed the PRD (proline rich domain) whereas the C-terminal half is typically more polar and has been called the MFMR (multifunctional mosaic region). This family may be composed of three sub-families called A, B and C classified according to motif composition. Some of these motifs may be involved in mediating protein-protein interactions. The MFMR region can contain a nuclear localisation signal in bZIP opaque and GBF-2. The MFMR also can contain a transregulatory activity in TAF-1. The MFMR in CPRF-2 can contain cytoplasmic retention signals.

A biomass-modulating polypeptide can contain a bZIP_{—}1 transcription factor domain. The basic-leucine zipper (bZIP) transcription factors of eukaryotic cells are proteins that contain a basic region mediating sequence-specific DNA-binding followed by a leucine zipper region required for dimerization.

A biomass-modulating polypeptide can contain a bZIP_{—}2 basic region leucine zipper domain. The basic-leucine zipper (bZIP) transcription factors of eukaryotic cells are proteins that contain a basic region mediating sequence-specific DNA-binding followed by a leucine zipper region required for dimerization.

A biomass-modulating polypeptide can contain an epimerase domain. An epimerase domain is typical of a family of proteins that typically utilize NAD as a cofactor. The proteins in this family can use nucleotide-sugar substrates for a variety of chemical reactions. The proteins in this family can use nucleotide-sugar substrates for a variety of chemical reactions.

Amino acid sequences for certain biomass-modulating polypeptides discussed above and domains indicative of biomass-modulating polypeptides, are described in more detail in U.S. Application Ser. No. 61/097,789.

A biomass-modulating polypeptide can encode a Dof transcription factor polypeptide. Dof transcription factors belong to a family of DNA binding proteins found in diverse plant species. Members of the Dof family comprise a Dof domain, which is characterized by a conserved region of about 50 amino acids with a C2-C2 finger structure associated with a basic region. See, e.g., Proc. Natl. Acad. Sci. USA 101:7833-7838 (2004).

Other Sequences of Interest

Other sequences of interest that can be used in the methods described herein include, but are not limited to, sequences encoding genes or fragments thereof that modulate cold tolerance, frost tolerance, heat tolerance, drought tolerance, water used efficiency, nitrogen use efficiency, pest resistance, biomass, chemical composition, plant architecture, and/or biofuel conversion properties. In particular, exemplary sequences are described in the following applications which are incorporated herein by reference in their entirety: US20080131581, US20080072340, US20070277269, US20070214517, US 20070192907, US 20070174936, US 20070101460, US 20070094750, US20070083953, US 20070061914, US20070039067, US20070006346, US20070006345, US20060294622, US20060195943, US20060168696, US20060150285, US20060143729, US20060134786, US20060112454, US20060057724, US20060010518, US20050229270, US20050223434, US20030217388, WO 2011/011412, WO 2010/033564, and WO2009/102965.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in sorghum is obtained, using appropriate codon usage bias tables.

### V. SORGHUM BREEDING

Fertile transgenic sorghum plants made by methods described herein typically are entered into a plant breeding program. Techniques suitable for use in a sorghum breeding program include, without limitation, backcrossing, mass selection, pedigree breeding, bulk selection, crossing to another population and recurrent selection. These techniques can be used alone or in combination with one or more other techniques in a breeding program. For example, each identified plant can be selfed or crossed to a different plant to produce seed that can be germinated to form progeny plants. At least one such progeny plant can be selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the transgene. In most breeding programs, analysis for the particular polymorphic allele will be carried out in each generation, although analysis can be carried out in alternate generations if desired. Progeny of a transgenic sorghum plant refers to descendants of a particular plant or plant line. Progeny of an instant plant include seeds formed on F₁, F₂, F₃, F₄, F₅, F₆ and subsequent generation plants, seeds formed on BC₁, BC₂, BC₃, and subsequent generation plants, and seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

The development of sorghum hybrids includes the development of homozygous inbred lines, the crossing of these lines, and the evaluation of the crosses. Pedigree breeding methods, and to a lesser extent population breeding methods, are used to develop inbred lines from breeding populations. Breeding programs combine desirable traits from two or more inbred lines into breeding pools from which new inbred lines are developed by selfing and selection of desired phenotypes. The new inbreds are crossed with other inbred lines and the hybrids from these crosses are evaluated to determine which have commercial potential.

Pedigree breeding starts with the crossing of two genotypes, each of which may have one or more desirable characteristics that is lacking in the other or which complement the other. If the two original parents do not provide all of the desired characteristics, other sources can be included in the breeding population. In the pedigree method, superior plants are selfed and selected in successive generations. In the succeeding generations the heterozygous condition gives way to homogeneous lines as a result of self-pollination and selection. Typically, in the pedigree method of breeding five or more generations of selfing and selection is practiced. F₁ to F₂; F₂ to F₃; F₃ to F₄; F₄ to F₅, etc.

Backcrossing can be used to improve an inbred line. Backcrossing transfers a specific desirable trait from one inbred or source to an inbred that lacks that trait. This can be accomplished for example by first crossing a superior inbred (A) (recurrent parent) to a donor inbred (non-recurrent parent), which carries the appropriate genes(s) for the trait in question. The progeny of this cross is then mated back to the superior recurrent parent (A) followed by selection in the resultant progeny for the desired trait to be transferred from the non-recurrent parent. After five or more backcross generations with selection for the desired trait, the progeny will be heterozygous for loci controlling the characteristic being transferred, but will be like the superior parent for most or almost all other genes. The last backcross generation would be selfed to give pure breeding progeny for the gene(s) being transferred.

The production of doubled haploids can also be used for the development of sorghum plants with homozygosity at one or more loci. For example, a transgenic sorghum cultivar can be used as a parent to produce doubled haploid plants. Doubled haploids are produced by the doubling of a set of chromosomes (1 N) from a heterozygous plant to produce a completely homozygous individual. This process obviates the need for generations of selfing needed to obtain a homozygous plant from a heterozygous parent.

A hybrid sorghum variety is the cross of two inbred lines, each of which may have one or more desirable characteristics lacked by the other or which complement the other. The hybrid progeny of the first generation is designated F₁. In the development of hybrids only the F₁ hybrid plants are sought. The hybrid is more vigorous than its inbred parents. This hybrid vigor, or heterosis, can be manifested in many ways, including increased vegetative growth and increased yield.

The development of a hybrid sorghum variety includes: (1) forming “restorer” and “non-restorer” germplasm pools; (2) selecting superior plants from various “restorer” and “non-restorer” germplasm pools; (3) selfing the superior plants for several generations to produce a series of inbred lines, which although different from each other, each breed true and are highly uniform; (4) converting inbred lines classified as non-restorers to cytoplasmic male sterile (CMS) forms, and (5) crossing the selected CMS inbred lines with selected fertile inbred lines (restorer lines) to produce the hybrid progeny (F₁).

Because sorghum is normally a self pollinated plant and because both male and female flowers are in the same panicle, large numbers of hybrid seed can only be produced by using CMS inbreds. Inbred male sterile lines are developed by converting inbred lines to CMS. This is achieved by transferring the chromosomes of the line to be sterilized into sterile cytoplasm by a series of backcrosses, using a male sterile line as a female parent and the line to be sterilized as the recurrent and pollen parent in all crosses. After conversion to male sterility the line is designated the (A) line. Lines with fertility restoring genes cannot be converted into male sterile A-lines. The original line is designated the (B) line.

Flowers of the CMS inbred are fertilized with pollen from a male fertile inbred carrying genes that restore male fertility in the hybrid (F₁) plants. An important consequence of the homozygosity and homogeneity of the inbred lines is that the hybrid between any two inbreds will always be the same. Once the inbreds that give the best hybrid have been identified, the hybrid seed can be reproduced indefinitely as long as the homogeneity of the inbred parent is maintained.

A single cross hybrid is produced when two inbred lines are crossed to produce the F₁ progeny. Much of the hybrid vigor exhibited by F₁ hybrids is lost in the next generation (F₂). Consequently, seed from hybrid varieties is not typically used for planting stock.

Hybrid sorghum can be produced using wind to move the pollen. Alternating strips of the CMS inbred (female) and the male fertile inbred (male) are planted in the same field. Wind moves the pollen shed by the male inbred to receptive stigma on the female. Providing that there is sufficient isolation from sources of foreign sorghum pollen, the stigma of the male sterile inbred (female) will be fertilized only with pollen from the male fertile inbred (male). The resulting seed, born on the male sterile (female) plants is therefore hybrid and will form hybrid plants that have full fertility restored. In some embodiments, if the hybrid sorghum is used as forage or for biomass production, then it may be unnecessary to restore fertility.

A double cross hybrid is produced when two inbred lines are crossed to produce the F₁ progeny, which is then crossed with a third inbred line. Such hybrids typically exhibit greater variability than single cross hybrids. This variability can be an advantage in adaptability across environments.

A top cross is a cross between a selection, line, clone etc., and a common pollen parent which may be a variety, inbred line, single cross, etc. The common pollen parent is called the top cross or tester parent. This type of test cross involves mating a series of individuals to a common parent to produce half-sib or full-sib families for evaluation. The test can be used to determine the general combining ability of an individual. Typically, those individuals that perform well in the testcross evaluation are advanced to trials where they are evaluated in crosses with other selected individuals. In sorghum, a top cross is commonly an inbred variety cross. In some embodiments, where the top cross is between inbred lines, and the resulting hybrids evaluated exhibit desirable traits, there may be no need for further testing and development, for example, where the resulting hybrids have a high biomass phenotype. In some embodiments, where the top cross is between inbred lines, and the resulting hybrids evaluated exhibit sterility, there may be no need for further testing and development.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent but at the same time retain many components of the nonrecurrent parent by stopping the backcrossing at an early stage and proceeding with selfing and selection. For example, a sorghum line may be crossed with another sorghum line to produce a first generation progeny plant. The first generation progeny plant may then be backcrossed to one of its parent varieties to create a BC₁ or BC₂. Progeny are selfed and selected so that the newly developed variety has many of the attributes of the recurrent parent and yet several of the desired attributes of the nonrecurrent parent. This approach leverages the value and strengths of the recurrent parent for use in new sorghum varieties.

Therefore, in one embodiment, a method of making a backcross conversion of a sorghum hybrid is described. The method can include crossing a plant of a sorghum hybrid with a donor plant comprising a desired trait, selecting an F₁ progeny plant comprising the desired trait, and backcrossing the selected F₁ progeny plant to a plant of the sorghum hybrid. This method may further include obtaining a molecular marker profile of sorghum hybrid and using the molecular marker profile to select for a progeny plant with the desired trait and the molecular marker profile of sorghum hybrid. In one embodiment the desired trait is a mutant gene or transgene present in the donor parent.

Mutation breeding is another method of introducing new traits into a plant (e.g., a hybrid). Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays, Gamma rays (e.g., cobalt 60 or cesium 137), neutrons (product of nuclear fission by uranium 235 in an atomic reactor), Beta radiation (emitted from radioisotopes such as phosphorus 32 or carbon 14), or ultraviolet radiation (preferably from 2500 to 2900 nm), or chemical mutagens (such as base analogues (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques. Details of mutation breeding can be found in “Principles of Cultivar Development,” Fehr, Macmillan Publishing Company (1993). In addition, mutations created in other sorghum plants may be used to produce a backcross conversion of a sorghum hybrid that comprises such mutation.

Sorghum breeding methods can include the use of genotyping techniques for marker-assisted breeding methods. Suitable genotyping techniques include Isozyme Electrophoresis, Arbitrarily Primed Polymerase Chain Reaction (AP-PCR), DNA Amplification Fingerprinting (DAF), and Sequence Characterized Amplified Regions (SCARs).

Genetic polymorphisms that are useful in such methods include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. For example, PCR techniques can be used to enzymatically amplify a genetic marker associated with a nucleotide sequence conferring a specific trait (e.g., nucleotide sequences described herein). PCR can be used to amplify specific sequences from DNA as well as RNA, including sequences from total genomic DNA or total cellular RNA. When using RNA as a source of template, reverse transcriptase can be used to synthesize complementary DNA (cDNA) strands. Various PCR methods are described, for example, in PCR Primer: A Laboratory Manual, Dieffenbach and Dveksler, eds., Cold Spring Harbor Laboratory Press, 1995.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. See Winn, et al. (2009) Int. J. Plant Genomics (2009):471853, Epub. 2009. The markers can also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses. Sorghum DNA molecular marker linkage maps have been constructed. See, Paterson, Int. J. Plant Genomics (2008) 2008:362451; Rouline A., et al., BMC Evol. Biol. (2009) 9:58; Paterson, et al., Nature (2009) 457(7229): 551-556; Sasaki, et al., Nature (2009) 457(7229): 547-548.

### VI. ARTICLES OF MANUFACTURE

A plant seed composition can contain a plurality of F₁ hybrid transgenic sorghum seeds described herein. The proportion of such seeds in the composition is from 70% to 100%, e.g., 90%, 91%, 92%, 93%, 94%, 95%, 96%, 97%, 98%, or 99% to 100%. The remaining seeds in the composition are typically seeds of one of the parents of the F₁, and the proportion of parent seeds is less than 5%, e.g., 0% to 0.5%, 1%, 2%, or 4%. The proportion of seeds in the composition is measured as the number of seeds of a particular type divided by the total number of seeds in the composition. When large quantities of a seed composition are formulated, or when the same composition is formulated repeatedly, there may be some variation in the proportion of each type observed in a sample of the composition, due to sampling error. In the present invention, such sampling error typically is about ±5%.

Typically, seeds are conditioned and bagged in packaging material by means known in the art to form an article of manufacture. Such a bag of seed preferably has a package label accompanying the bag, e.g., a tag or label secured to the packaging material, a label printed on the packaging material or a label inserted within the bag. The package label indicates that the seeds therein are F₁ hybrid sterile transgenic sorghum seeds. The package label may indicate that plants grown from such seeds are suitable for making an indicated preselected polypeptide. The package label also may indicate the seeds contained therein incorporate transgenes that provide biological containment or confinement of plants grown from the seeds.

The commercial production of seeds for growing sorghum plants normally involves four stages, the production of breeder, foundation, certified and registered seeds. Breeder seed is the initial increase of seed of the variety which is developed by the breeder and from which foundation seed is derived. Foundation seed is the second generation of seed increase and from which certified seed is derived. Certified seeds are used in commercial crop production and are produced from foundation or certified seed. Foundation seed normally is distributed by growers or seedsmen as planting stock for the production of certified seed.

### VII. USES AND ADVANTAGES

Sorghum hybrids provided herein have various uses in the food, agricultural, and energy production industries (e.g., biofuels such as ethanol). For example, sorghum plants described herein can be used to make animal feed and food products. The sorghum plants described herein can have reduced susceptibility to ergot fungal infections as preventing development of an ovary, such as by affecting a developmental stage such as spikelet meristem identity, establishment of floral meristem identity, or floral organ initiation, development, or function can prevent the fungal spores from infecting the stigma.

The F₁ sorghum hybrids described herein advantageously can be produced without the need to apply any sort of chemical inducer or chemical ligand to induce sterility or reduced fertility.

Sorghum plants described herein can be grown in large fields (e.g., 50 to 10,000 acre fields) to obtain harvestable biomass. For example, the sorghum plants provided herein can be grown in fields of 100 acres or more at locations suitable for sorghum growth such as southern United States, Brazil, and Mexico.

In one embodiment, the stalks of sorghum plants described herein are harvested and processed, e.g., extracted using pressing and/or milling techniques, to obtain sorghum juice. For example, the stalks can be harvested by hand or mechanical harvesters, and then crushed and pressed with a horizontal or vertical mill to extract the juice. One objective of the pressing and/or milling processes is to extract the largest possible amount of juice from the sorghum biomass. Another objective is to produce bagasse with a low moisture content to be burned as a boiler fuel for electricity generation, thereby allowing a production plant to be self-sufficient in energy.

Sucrose, i.e., table sugar, can be produced from the juice using techniques including filtering, clarifying, decolorizing, and repeated concentration and crystallization. In some embodiments, table sugar is produced by blending sweet sorghum juice with sugarcane juice prior to crystallization, thereby increasing the total yield of table sugar.

In other embodiments, the sugars in the juice can be fermented to produce a biofuel. For example, the juice can be filtered and used in a fermentation reaction to produce a biofuel. Examples of biofuels include, without limitation, biodiesel, methanol, ethanol, butanol, linear alkanes (C5-C20), branched-chain alkanes (C5-C26), mixed alkanes, linear alcohols (C1-C20), branched-chain alcohols (C1-C26), linear carboxylic acids (C2-C20), and branched-chain carboxylic acids (C2-C26). In some cases, the methods and materials provided herein can be used to make other chemical compounds such as ethers, esters, and amides of the aforementioned acids and alcohols, as well as other conjugates of these chemicals. In some cases, one or more of these compounds can be chemically converted into other high value and/or high volume chemicals.

Any appropriate microorganism can be used to produce biofuel in a fermentation reaction. For example, one or more microorganisms designed to produce ethanol can be used in fermentation reactions with sorghum juice to produce ethanol-containing reaction products. In some cases, a microorganism useful for producing one or more biofuels as described herein is from a genus such as Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, and Saccharomyces. For example, ethanologenic yeast can be used in a fermentation reaction containing sorghum juice to produce ethanol.

Any appropriate fermentation process can be used to produce biofuel using sorghum juice. For example, batch, fed-batch, or continuous fermentation processes can be used to produce a biofuel using sorghum juice. A batch fermentation process can include adding sorghum juice substrate, fermentation organism(s) and culture medium at the beginning of the fermentation and not replenishing once fermentation has begun. In some cases, one or more culture parameters, e.g., pH and oxygen concentration, are monitored and adjusted during the fermentation process.

In some cases, a fed-batch fermentation process can be used to produce biofuel using sorghum juice obtained from sorghum plants provided herein. A fed-batch fermentation process is similar to a batch fermentation process except that substrate is added, and optionally culture medium nutrients, at intervals as fermentation progresses. In some cases, one or more culture parameters, e.g., pH, dissolved oxygen concentration, and/or carbon dioxide to oxygen ratio, are monitored and adjusted during the fermentation process. Fed-batch fermentation processes can allow users to control the amount of substrate within the fermentation reaction.

Continuous fermentation processes also can be used to produce biofuel using sorghum juice obtained from sorghum plants provided herein. A continuous fermentation process can be an open system in which a defined fermentation medium containing sorghum juice material is continuously added to a bioreactor and an amount (e.g., an equal amount) of conditioned media is continuously removed for subsequent processing. Continuous fermentation can often be performed such that the fermentation organism is maintained at a high cell density and in a prolonged exponential growth phase, resulting in higher productivity than batch fermentation.

Examples of batch, fed-batch, and continuous fermentation processes that can be used to produce biofuel using sorghum juice obtained from plants provided herein are described elsewhere (Thomas D. Brock in Biotechnology: A Textbook of Industrial Microbiology, Second Edition (1989) Sinauer Associates, Inc., Sunderland, Mass.; and Deshpande, Mukund V., Appl. Biochem. Biotechnol., 36:227 (1992)).

Any appropriate fermentation media containing sorghum juice can be used in a fermentation reaction to produce biofuel. In some cases, fermentation media used to produce biofuel as described herein can contain sorghum juice as the primary carbon source (e.g., primary source of glucose, fructose, sucrose, mannose, or other sugars). In some cases, one or more other carbon sources can be used in combination with sorghum juice provided herein to form fermentation media for producing biofuel. For example, sorghum juice obtained from sorghum plants provided herein can be combined with sugarcane juice (garapa) to form fermentation media for producing biofuel. In some cases, one or more other components such as minerals, salts, cofactors, and buffers can be included within fermentation media to promote culture growth and/or biofuel production. Examples of commercially available broths that can be used in combination with sorghum juice material to create fermentation media include, without limitation, Luria Bertani (LB) broth, Sabouraud Dextrose (SD) broth, and Yeast medium (YM) broth.

Any appropriate culture conditions can be used to perform fermentation reactions designed to produce biofuel using sorghum juice. For example, fermentation cultures can be grown or maintained at a temperature in the range of about 25° C. to about 40° C. and at a pH in the range of pH 5.0 to pH 9.0 (e.g., a pH in the range of 6.0 and 8.0, of 6.5 and 7.5, or 6.5 and 7.0). A fermentation reaction can be performed under aerobic, microaerobic, or anaerobic conditions.

In some cases, biofuel production can be monitored during a fermentation reaction or can be assessed when the fermentation reaction is completed. Any appropriate method can be used to assess biofuel production. For example, high performance liquid chromatography (HPLC) or gas chromatography (GC) can be used to measure biofuel production.

Once produced, biofuel can be isolated from the fermentation product. For example, techniques such as centrifugation, filtration, decantation, or combinations thereof can be performed to remove solids from the fermentation product. Once most or all of the solid material is removed, biofuel present within the remaining material can be isolated by, for example, techniques such as distillation, liquid-liquid extraction, dehydration, membrane-based separation, or combinations thereof. In some cases, molecular sieves, distillation techniques, azeotropic distillation techniques, centrifugation, vacuum distillation, or combinations thereof can be used to separate biofuel (e.g., ethanol) from water and/or fermentation byproducts. For example, water can be removed from an azeotropic ethanol/water mixture obtained from a fermentation reaction by azeotropic distillation to result in hydrous ethanol having about 95 to about 96.5 percent ethanol and about 3.5 to about 5 percent water. Azeotropic distillation can include adding benzene or cyclohexane to an ethanol/water mixture. When these components are added to the mixture, they can form a heterogeneous azeotropic mixture in vapor-liquid-liquid equilibrium. This can be distilled to produce anhydrous ethanol at the bottom of a column and a vapor mixture of water and cyclohexane/benzene. When condensed, the material can become a two-phase liquid mixture. In some cases, an extractive distillation process that involves adding a ternary component that increases the volatility of ethanol can be performed. Distillation of the ternary mixture can result in anhydrous ethanol on the top stream of a column.

In some cases, dehydration methods such as those involving molecular sieve techniques can be used to remove water from a biofuel. For example, ethanol vapor under pressure can be passed through a bed of molecular sieve beads. The pore size of the beads can be designed to allow absorption of water while excluding ethanol. After a period of time, the bed can be regenerated under vacuum or through the flow of inert gas (e.g., N2) to remove absorbed water. In some cases, two or more beds of beads can be used. In such cases, one can be used to absorb water, while the other one is undergoing regeneration. In some cases, the use of molecular sieve techniques can be performed in a manner that does not involve the use of distillation techniques.

In some cases, production of ethanol for biofuel involves denaturation of the ethanol. Ethanol can be denatured by, for example, combining it with natural gasoline, unleaded gasoline, or gasoline blend stocks. Corrosion inhibitors such as Ashland Amergy ECI-6 or Petrolite Tolad 3222 can be added to fuel ethanol if desired. Ethanol for fuel use can meet the specifications of ASTM D4806 (e.g., ASTM D4806-09). In some cases, the ethanol meets the specifications of ASTM D5453-93 for sulfur content, the specifications of ASTM D5580-95 for benzene or aromatic content, and/or the specifications of ASTM D6550-00 for olefin content. In some cases, ethanol for fuel use, produced as described herein, can meet Brazilian specification ANP#36 for hydrous ethanol or anhydrous ethanol.

In some cases, biomass remaining after extraction of juice (e.g., bagasse such as low moisture bagasse) or biomass not used for juice extraction can be used as a source of cellulosic material. Such cellulosic material can be used in fermentation reactions designed to metabolize cellulose and/or other sorghum biomolecules in order to produce biofuel or can be used in combustion reactions designed to produce heat for use in energy production.

The invention is further described in the following example, which does not limit the scope of the invention described in the claims.

### EXAMPLE

### Example 1

### Transgenic Sorghum Plants Having Increased Sucrose Purity

Sorghum germplasm of the Wheatland variety was transformed according to the methods of PCT/US11/22738 using an RNAi vector designed to inhibit expression of Frizzy Panicle (FZP) (SEQ ID NO:1). A T₀ transgenic sorghum plant was identified that had significant reduction in seed set, i.e., fewer than 10 seeds on a full panicle (wild type panicles typically hold 200 or more seeds). All viable seeds were harvested from the transgenic plant, planted in soil, and allowed to grow into mature T₁ plants. Eight of the T₁ plants reached maturity at the same time as measured by heading date and anthesis date. Five of these eight plants were significantly reduced in fertility (less than 20% fertility). Three of the plants were phenotypically wild type.

Stems were harvested from all eight plants at the milk-soft dough stage of development (about three weeks after full panicle emergence). Juice was pressed from the stems then analyzed by high performance liquid chromatography (HPLC) to determine the sugar profile. In some instances, frozen juice samples were thawed at room temperature for 1 to 2 hours before analyzing. Juice samples were homogenized using a standard mini vortexer for 5-10 seconds. One (1) mL of homogenized sweet sorghum juice was transferred to a 2 ml microcentrifuge tube and centrifuged at 10,000 rpm for 5 minutes at 4° C. Four hundred (400) μL of the supernatant was removed using a 1 mL syringe (BD, Catalog No. 309602) and filtered using a 0.2 μm filter (Life Sciences, Catalog No. PN 4540). The filtered sample was placed into 500 μL HPLC vials (Alltech, Catalog No. 98842) and analyzed using the Agilent 1100 series HPLC system. The samples were used directly (without dilution) or diluted based on the Brix values measured using a pocket refractometer (Atago, Model Name PAL-3, Catalog No. 3830). Samples were diluted with HPLC grade water (EMD, Catalog No. WX0008-1) such that the concentration of each sugar fell within the validated range of the analytical method (Sucrose: 10˜160 mg/ml; Glucose: 1˜19 mg/ml, Fructose: 1˜19 mg/ml).

Parameters for the HPLC included:

Column: Aminex® HPX-87P (Biorad Aminex HPX-87P, Catalog No. 1250098)

Mobile Phase: Water (EMD, catalog Number: WX0008-1)

Flow rate: 0.6 ml/min

Column Temperature: 80° C.

Detector: Corona CAD

Software used for Data analysis: Chemstation (Agilent Technologies)

As shown in Table 3, average sugar density (i.e., sugar density is mg of total sugar content/mL of juice, total sugar content refers to total of sucrose, glucose, and fructose,) and average sugar purity (i.e., sucrose/total sugar content) were higher in the transgenic plants with reduced fertility.

TABLE 3

| | | Ave | | | Ave | | |
|---|---|---|---|---|---|---|---|
| | N | Sugar | | | Sugar | | |
| Sample | (sample #) | Density | SD | SE | Purity % | SD | SE |
| Reduced | 5 | 139.1 | 8.2 | 3.7 | 97.6% | 0.5% | 0.2% |
| fertility | | | | | | | |
| Fertile | 3 | 89.0 | 25.5 | 14.7 | 94.3% | 1.6% | 0.9% |

SD = Standard Deviation; SE = Standard Error

### Example 2

The transgenic Wheatland plants of the previous example were crossed with sweet sorghum of the Umbrella variety. The F₁ hybrid seeds were grown and the measurements shown in Table 4 were taken at the following stages: booting stage, milk/soft dough (3 weeks post-booting), and black layer (6-weeks post booting). The controls were the segregating non-transgenic F₁ plants. As shown in Table 4, average total sugar content, average sugar purity, and sugar density were higher in the hybrid plants with reduced fertility in the milk/soft dough and black layer stages. Average sugar purity and sugar density also were higher in the booting stage in the hybrid plants.

TABLE 4

| | | Sugar Density (mg/mL) | | | Sugar Purity | | | Juice volume (mL/3 plants) | | Total Sugar Content (g/3 plants) | | |
|---|---|---|---|---|---|---|---|---|---|---|---|---|
| Stage | Phenotype | Avg | Std err | Increase | Avg | Std err | Increase | Avg | Std err | Avg | Std err | Increase |
| Booting | Control | 64.8 | 6.3 | | 69.0% | 3.9% | | 515.0 | 15.0 | 33.4 | 3.7 | |
| stage | F1 hybrid | 69.7 | 1.4 | 7.6% | 79.1% | 1.6% | 14.7% | 450.0 | 56.9 | 31.2 | 3.4 | −6.6% |
| Milk/soft | Control | 131 | 1.2 | | 90.2% | 0.7% | | 611.7 | 103.1 | 80.1 | 13.4 | |
| dough | F1 hybrid | 144.9 | 3.3 | 10.6% | 91.8% | 1.2% | 1.8% | 690.0 | 28.9 | 100.1 | 5.7 | 25.0% |
| Black | Control | 134.0 | 4.4 | | 92.6% | 1.4% | | 715.0 | 50.7 | 95.9 | 8.2 | |
| layer | F1 hybrid | 151.3 | 11.4 | 12.9% | 94.9% | 0.6% | 2.5% | 717.5 | 60.7 | 107.5 | 6.5 | 12.1% |

| | Estimated total seed | | | Seed Wt | |
|---|---|---|---|---|---|
| | Average | Std Err | % fertility | Average | Std Err |
| Control | 1376 | 134.6 | 100% | 31.0 | 1.4 |
| F1 hybrid | 497 | 23.8 | 36% | 32.7 | 0.9 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A sorghum plant, said plant comprising an exogenous nucleic acid comprising a regulatory region operably linked to a plant sterility sequence, wherein said plant sterility sequence affects a developmental stage selected from the group consisting of i) spikelet meristem identity, ii) establishment of floral meristem identity, and iii) floral organ initiation, development, or function; wherein the stalk of said sorghum plant has a sucrose purity that is higher at maturity than that of a corresponding control plant that lacks said exogenous nucleic acid.

2. The plant of claim 1, wherein said plant has reduced fertility.

3. The plant of claim 1, wherein said stalk of said sorghum plant has an increased total sugar content at maturity relative to that of said corresponding control plant.

4. The plant of claim 3, wherein said stalk has a total sugar content that is increased by 12% or more relative to a corresponding sorghum plant that lacks said exogenous nucleic acid.

5. The plant of claim 1, wherein said stalks have a total sugar content that is increased by 25% or more relative to a corresponding sorghum plant that lacks said exogenous nucleic acid.

6. The plant of claim 1, wherein said stalks have a total sugar content that is increased by 12 to 25% relative to a corresponding sorghum plant that lacks said exogenous nucleic acid.

7. The plant of claim 1, wherein said stalks have a total sugar content that is increased by 40% to 60%, relative to a corresponding sorghum plant that lacks said exogenous nucleic acid.

8. The plant of claim 1, wherein said plant is an F₁ hybrid.

9. The plant of claim 1, wherein said plant is male sterile.

10. The plant of claim 9, wherein said plant exhibits cytoplasmic male sterility (CMS).

11. A plurality of F₁ transgenic sorghum seeds, said seeds comprising an exogenous nucleic acid comprising a promoter operably linked to a plant sterility sequence, wherein said plant sterility sequence affects a developmental stage selected from the group consisting of i) spikelet meristem identity, ii) establishment of floral meristem identity, and iii) floral organ initiation, development, or function; wherein F₁ sorghum plants grown from said F₁ seeds express said plant sterility sequence, and wherein stalks of said F₁ sorghum plants have a sucrose purity that is higher at maturity than that from corresponding control plants that lack said exogenous nucleic acid.

12. The sorghum seeds of claim 11, wherein said stalks of said F₁ sorghum plants have an increased total sugar content at maturity relative to that of said corresponding control plants.

13. The sorghum seeds of claim 11, wherein said stalks of said F₁ sorghum plants have a total sugar content that is increased by 12% or more relative to that of said corresponding control plants.

14. The sorghum seeds of claim 11, wherein said stalks of said F₁ sorghum plants have a total sugar content that is increased by 25% or more relative to corresponding sorghum plants that lack said exogenous nucleic acid.

15. The sorghum seeds of claim 11, wherein said stalks have a total sugar content that is increased by 12 to 25% relative to a corresponding sorghum plant that lacks said exogenous nucleic acid.

16.-59. (canceled)

60. A process for making a biofuel, wherein said process comprises:
(a) harvesting biomass from sorghum plants grown from F₁ seeds of claim 11 to obtain harvested sorghum biomass;
(b) extracting sorghum juice from said harvested sorghum biomass to obtain extracted juice comprising sugar;
(c) using said sugar of said extracted juice in a fermentation reaction to produce a fermentation product comprising a biofuel; and
(d) isolating said biofuel from said fermentation product to obtain a composition comprising said biofuel.

61. A process for making a biofuel, wherein said process comprises:
(a) harvesting biomass from sorghum plants of claim 1 to obtain harvested sorghum biomass;
(b) extracting sorghum juice from said harvested sorghum biomass to obtain extracted juice comprising sugar;
(c) using said sugar of said extracted juice in a fermentation reaction to produce a fermentation product comprising a biofuel; and
(d) isolating said biofuel from said fermentation product to obtain a composition comprising said biofuel.

62. The process of claim 60, wherein said biofuel is ethanol.

63. The process of claim 60, wherein said composition comprises anhydrous ethanol.

64. The process of claim 60, wherein said biomass comprises stalks of said sorghum plants.

65.-70. (canceled)


SEQUENCE LISTING (38 sequences)

SEQ ID NO: 1 - DNA, 984 bp, Sorghum bicolor
  misc_feature: Ceres Annot ID no. 8657227
atgatgaatacccgaggcagtggcagtggcagcagcagcggcagcaaccagaccatgatggccttctccgaccatcctccgaagccggcgacggcagcgtcaggcgggcagccgcagccgtccccgccgtcgtctccgagcgagcgtccgccagccggccgtgggcgccggcgcgcgcaggagcccgggcggttcctcggcgtgcgtcgccggccgtggggacggtacgcggcggagatacgcgaccccacgaccaaggagcggcattggctgggcacgttcgacaccgcgcaggaggcggcgctggcctacgaccgcgcggcgctgtccatgaagggcgcgcaggcgcgcaccaacttcgtctacacgcacgccgcgtacaactacccgcccttcctggcgccgttccaccacgggcagcagccgtcgtcgtacgcgcacgcgccgtcgtccgtgcagcaccagtacggcggcggagtgggcgccggcgcgccgcacattgcctcgtacgggcaccaccactaccaccaccaggccggctcggcgagcgctgcagcagctggcggcgcgtcgtcgggcgagtgctcgtcgacgatgccggtgcccgtggaacgcgccgacggcacgctgctgctggaccgcagcggcggcggtcaccaccaccaccacgagttcctgttcgcgagcgcggacgacaactccgggtacctgagcagcgtggtcccggagagctgcctccggccgaggagcagcgcggcggcggtggaggacctgcggcggtactcggacgcggacgcctacgggatgggcatggggctccgggaggacgtggacgaccttgcccagatggtggccgggttctggggcggcgctggcgacgcggaccagctgtgcgggttcccgtccggcggcgccggggacagcatggtggcgtcgtcgcagggctctgacggctactcgcccttcagcttcctctcccactga

SEQ ID NO: 2 - DNA, 1686 bp, Sorghum bicolor
  misc_feature: Ceres Annot ID no. 8658024
atggtcgcgctctgcttcctcctcagctacgtcttctcggccctcaccgtcgacccgcacccgcccacgctcgccgcctcagcggcgccgcctccgaccgcgtcgtcgtcgtccgtcgtcatcgacgacatcatccgagtcctcgccgacattaataccaccgcagcgcgcatccgcacggacgccgaggccacggcgcgcgcgtccaccgacttcggcaccaacgtgaccgtggacgcagcgcggcgcccggcggccgtgttctacccgtcgtgcgcggccgacatcgccgcgctgctgcgggcgtccagcgccagcgccacgccgttcccggtctccgccaggggccgcggccactccacccggggccaggccacggcccccggcggcgtcgtcatcgacatggcgtcgctcgccgtcgcagcagggcgccaccacaggctcgccgtgtcggtggacgggcgctacatcgacgccggcggcgagcagctgtgggtggacgtgctgcacgccgcgctggcgcacggcctcacgccgcggtcctggacggactacctccacctcaccgtcggcggcacgctctccaacgccggcatcagcgggcaggcgttccgacacggcccgcagatatccaacgtcctcgagctcgacgtcgtcacgggaacaggtgacatggtcacgtgctccaagcacaaggacgctgacctcttcgacgccgtgctgggagggctggggcagttcgggatcataacgcgcgcgcggatcccgctggcgccggcgccggccagggcgcgctgggtgcggctcctctacaccgccgccgcggacctcacggccgaccaggagcggctcatcgacgacggcggcgcgctggccgggctcatggactacgtcgagggctccgtcctcaccgacttccagggccagggcctcatcggcagctggcgctcgcagccgccgtcgtccttctactcgacggccgacgccgcgcgcatcgccgcgcttgccaaggaggccgccggcgtcctgtactgcctcgagggcgcgctgtactacggcggcgccagcgacacgaccgccgcagacgttgacaaggagctgcggtacgcgcgggggttcgcgttcgtgcaggacgtgtcgtacgtgcagttcctagaccgcgtgagcgccggcgagcgcaagctccgcggcgagggcctctgggacgtgccgcacccgtggctcaacctcttcctcccgcgctccagcatcctcgacttcgccgcgggcgtcttccacggcgtcctgctccgcggcggcggcggcggcggccccgtgctcgtctaccccatgaaccggggcaagtgggacagcgccacgtcggcggtgctccccctccccgaggacgatgaggacgacgacgaggtgttctatacggtggggatcctgcggtcggcggtggcggacggcgacatgcggcggatggaggagcagaacgcggaggtggcgcggttctgcgaggccgccggcatcccgtgcacgcagtacctggcctactacacgacgcaggcggagtgggcggcgcggcacttcgggacccgcaggtgggacaccttcctccgccgtaagaggaaatacgaccccatggcaatcctgtcgcggggccagaggattttctcgtacccacttgccggcctgatgatatga

SEQ ID NO: 3 - DNA, 1167 bp, Sorghum bicolor
  misc_feature: Ceres Annot ID no. 8654369
atgggggaggaggtagcggcggcggcggcggtggtgctggagccgccacggcccaagtcgccgccgaggtacccggacctgtgcggccgccggcggctgcagctggagctccagatcctcaaccgcgagatcgacttcctcaaggacgagctacaatcactagaaggagttccaccagtttctagaagttgtaaagagtatgcactaaagaagaagacgcacagatcttgccgccttttttggtggatcagatcaaaattgtgcatatgcgtgtcatggttctgctgctcctgccattgcttgcccaactgcaaaagaccatgctgcttggattgctcctgctgctcatgcccagatctgtcatgctgcaagcccagctgcaaatcatgcaacaagccctgctcttggcccaacagctgttcatgttgtgacacaccatgctgcaagccagattgcccatcctgtagttcgagctgcagctcgtgctgcaatctgtcgtgttgcaacccgaactgtagctcgtgctgcacctgcaatccatcatgctgtaaaccaaactgcaactcgtgctgcagaccaaactgtagctcgtgctgcaatccgtcatgctgcaaacctaactgcggctcatggtggaaaccgagctgcagctgcttcaaggccccctcgtgctgcaaactccagtgcagccccaactgctgcacttgcagcctcccgcgctgctccggctgcaacccctgcggcagttgcaagcagtgttgctcatgccccaccgactgctgcaactgcaagccaagctgcggctgtttcagcgcgcaatgttgcagctgtgcggcatgctgctcctcgtgcttaagctgcttcagttgcttcggttgcttcaagtccttcaagtgctccaacctgttcgggtgctgctcctgcaagcagtgcttcaagtgccagtcgtcgtgctgcaagggtgcgccatcctgctgcaagtgccagtcgtcgtgctgtgagggtgaagacggcagcagctgctgccggaggtcatgctgcagtgttccgaagccggcttgccctgggtgctcgtgcgggtgcgtttggtcctgcaagaagtgtacagatgggtgtcgatgttctgggtgccgcaatccgtgctgtgccactggatgcttgtgttga

SEQ ID NO: 4 - DNA, 726 bp, Sorghum bicolor
  misc_feature: Ceres Annot ID no. 8632646
atgggccgcgggaaggtggtgctgcagcggatcgagaacaagatcagccggcaggtgaccttcgccaagcgccggaacggcctgctcaagaaggcctacgagctctccatcctctgcgacgccgaggtcgcgctcgtcctcttctcccacgccggccgcctctaccagttctcatcctcctccaatctgcttaagactctagagcgataccagaggtacatctatgcttcagctgatgctgcagtgccatctagcgatgagatgcagaataactatcaagaatatgtgaacctgaaggcacgagttgaggttttacaacactcacaaaggaatcttcttggtgaagatctggctccacttagccctagtgaacttgaccagcttgagagtcaagtagacaagaccttgaagcaaataagatcaagaaagactcaagtgttacttgatgaactttgcgacttaaagagaaaggaacaaatgctgcaagatgcaaacagggttctgaaaaggaagctggacgagtttgaggcagaggctgcttctcccccgcagctagcgtggcaagacgggggcggcatgttgtcccatgaccctccacagccagaacatttcttccaggctctggagagcaacccatctctgcaacctacataccatacgatggacatgaaccagcagccagtgccatcgccgggcagctgctaccctcctgattggatggcttag

SEQ ID NO: 5 - DNA, 1620 bp, Sorghum bicolor
  misc_feature: Ceres Annot ID no. 8645308
atggcttcgcctgcgataccattcgcccctctcacgtcccatcgcgccgttccctttgtcttggggtgtcctcctccatggccaccgcgcccgccgcctgccgctcccggacggccgccgcgccccgacgctgctgcagctgctagactgctagaggaggaggccagggcaggcagctcgcgagctcgatcgcctgccggccggcccgagctggagtcgatggtgctggatctcaatgccgagtcgccgacggccggctcggcctcggccacctcgagctccagcggcgtcttccggttcgacctgctcggagggacacccgacgaggagggttgctcgccctcgccgcccgttgtgacgcgccagctcttccccttgccgtcgtacccggacgctgctgcagctcccaccgccgcctcgaacgggtcgccgccgccgccgcaggcggcagggccatgggcgcgccgcgcagcggatctcgtggcgccggcgctgggacagggacagggacagggggcggtggttatgcctgcgccttcgtcgccgcccgcggctgtgtctccagccgccgggaagaagagccgacgggggcccaggtcgcggagctcgcagtacaggggcgtcaccttctacaggaggaccggccgctgggagtcacacatctgggactgcgggaagcaggtctatctgggtggatttgatactgcccacgccgcagcaagggcctatgatcgtgctgcaatcaagttcagaggccttgacgcagacatcaatttccagttgaaggactatgaggatgatttgaagcagatgaagaattggaccaaggaggaatttgttcacatactccgacggcaaagcactgggtttgccagggggagctcaaagtaccgcggtgtgacactgcacaagtgtggtcggtgggaagctcggatgggtcagcttctgggaaagaagtacatctaccttgggctgtttgacagtgaaatcgaagcagcaagagcgtatgacagggcagccattcgcttcaacggaccggatgctgttactaacttcgattctagttcctatgatggagatgtcccacttccaacggcaatcgagaaagatgtggttgatggggacatccttgatctgaatttgaggatttcacaacctaatgtgcatgatctcaaaagtgatggtaccctgactggttttggattaggttgtaattctcctgaagcttcaagctccattgtttctcagccaataagccctcagtggcctgtgcatcctcacagcacaccgatgcaactccagcatccacatctgtatgcgtctccttgtccaggcttctttgtgaacctcagggaggcgcctatggaggaggagaaacgggcagagcgggcgggtcccgagccggcgttcccctcttgggcatggcaaacgcagggctcccctgccccgtttctccctgccactgctactgcagcatcatcaggattctctacagccgccaccaccaccggcgtggacgcggccacagccgcccgcagcgtgccgccgtccttgtccggtggcccccggcagttgttctccggctaccagctccagctccgcttccccccaaccgcctga

SEQ ID NO: 6 - DNA, 546 bp, Sorghum bicolor
  misc_feature: Ceres Annot ID no. 8642422
atgtacaaaacactcgagagataccgcagctccaactacagctcacaggaagtaaaagttccactggacagtgaaattaactaccaggattatctgaagttgaggacaagagttgaatttcttcaaactacacaaagaaatattcttggtgaggatctgggtccacttagcatgaaggaacttgagcagctggagaaccaaatagagacatccctgaagcagatcaggtcaagagagaatcaaatgttacttgatcagctctttgatctcaaaagcaaggaacaagaattgcaggatctcaataaagacctaaggaaaaagctgcaagaaacaagtccagagaatgtgctccatgtttccagctgggaggaaggtgggcacagtggcgcaagtggtaatgttcttgatccttatcagggactccttcagcacccagagagtgatccttccctgcagattgggtaccatcaacaagcctacatggaccagctgaacaacgaggacatggcgggcccaaatgagcacggtcgatctggatggatatga

SEQ ID NO: 7 - DNA, 1296 bp, Sorghum bicolor
  misc_feature: Ceres Annot ID no. 8743976
atgatgcaccaccaccctcagttcctgcttccgcgcctccactcgccgtcgtcgtcctcctcgtctgaggcggtggacatggacccccgcgtgtggggccgactgccgcagccgctggtggaccgcgtgctggcgtgcctaccgacgccgtccttcctccgcctccgggccgcctgccgccgcttctgcaacctcatctactcgtcgccgttcctccactcccacctcctcctctccccgcacctccccttcttcgccttcgccgtcccttccgcggggtacctcctcctgctcgaccccaccaggccggaggcgccctcctggtcccgcctcccgctgccgctgcccgcggcgcccggcgctggccaccaggcgttctccccggcggccgcgtccgcgggcctgctcgcgttcctgtcggacgcgtccggccacaagacgctgctcctcgccaaccccatcacgcgcctcctcgcgccgctgccgctctgccccaccgcgcgcctctcccccaccgtcggcctcgccgccggacccacctccttcatcgccgtcatcgccggcgacgaccttgtgtcccccttcgcggtcaagaacatctccgccgacacgttagtcgccgatgccgcctccgtcccaccctccggtttctgggccccgagttccatcctgccccgcctctcctccctcgaccctcgcgccggcatggctttcgcctccggaaggttctactgcatgagctcgtcgccgttcgcggtgcttgtgttcgacgtggcgaccaacgtctggagcaaggtgcagccgccgatgaggaggttcctgcggtcgccggcgctcgtggagttcggcggcgggagggagcgcgaggcgagggtggcgctggtctccgccgtcgagaagagccgcctcagcgtgccacggagcgtgcgcgtgtggacgctgcgcggcgggagcaacggcggcagcggggcgtggaccgagatggcgcggatgccgcccgacgtgcacgcacagtttgcggcggccgagggcgggcgcgggttcgagtgcgcggcgcacggcgacttcgtggtgctggcgccacgcgggcccgcgatccccgtgctcgtgttcgactcgcgccgcgacgagtggcggtgggcgccgccgtgcccgtacccgccgtacgccggggggatcgccgcgggaggtgccgggttcagggtgttcgcttacgagccacgcctggcgacgccggccatcggcctcctggacgccacggcgccggcggcctttttgcatgggatgcagggctag

SEQ ID NO: 8 - DNA, 1176 bp, Sorghum bicolor
  misc_feature: Ceres Annot ID no. 8702677
atggatcccaacgacgccttctcggcggcgcacccgttccggtgggacctgggcccgccggcgcacgccgcgcctccgcctccgcctccgccgcagccagcgccagcgccagcgccgcagctgccgcctcacgcgccgctggtgtacgcggcgccgagggagctggaggacctggtggccggctacggcgtgcgcccgtccacggtggcgcggatctcggagctcgggttcacggcgagcacgctcctgggcatgacggagcgcgagctggacgacatgatggccgcgctcgcgggtctgttccgctgggacgtgctcctcggcgagcggttcggcctccgcgccgcgctgcgggccgagcgcggacgtgtcatgtccctcggcggccgcttccacaccgggagcaccttggatgccgcgtcacaggaagtgctgtccgacgagcgcgacgcggcggccagcggcggcacggtgatggccggcaagaagaagggtaacaaaggggtcggctcgaggaagggcaagaaggcgaggaggaagaaggagctgcggccactggacgtgctgggcgacgagaacgacggagacgaggacggcggcggcgggtcggagtcgacggagtcgtccgcgggcggctccggcggcggggagaggcagcgggagcacccgttcgtggtcacggagcccggcgaggtggcgagggccaagaagaacggcctcgactacctcttccatctgtacgagcagtgccgcgtcttcctcttgcaggtgcagtccattgctaagctgggcggccacaagtcccctaccaaggtgacgaaccaggtgttccggtacgccaagaagtgcggggcgagctacatcaacaagcccaagatgcggcactacgtgcactgctacgcgctgcactgcctggacgaggaggcctccaacgcgctgcgccgggcgtacaaggcccgcggcgagaacgtcggcgcctggaggcaggcgtgctacgcgccgctcgtcgagatcgccgcgcgccacggcttcgacatcgacgccgtcttcgccgcgcacccgcgactcgccatctggtacgtgcccaccaggctgcgccagctctgccaccaggcgcgggggaaccacgcccacgtccacgccgccgccggcttcccgccgccgccgatgttctag

SEQ ID NO: 9 - DNA, 744 bp, Sorghum bicolor
  misc_feature: Ceres Annot ID no. 8632648
atggggcgcgggaaggtgcagctgaagcggatcgagaacaagatcaaccgccaggtgaccttctccaagcgccgctcggggctgctcaagaaggcgcacgagatctccgtgctctgcgatgccgaggtcgcgcttatcatcttctccaccaagggcaagctctacgagtactccaccgattcatgtatggacaaaattcttgaacggtatgagcgttactcctatgcagaaaaggttctcatttcagcagaatctgaaactcagggcaactggtgccacgaatatagaaaactaaaggcgaaggtcgagacaatacagaaatgtcaaaagcacctcatgggagaggatcttgaaactttgaatctcaaagagcttcagcaactagagcagcagctggagagttcactgaaacatatcagaaccaggaagagccagcttatgctcgagtcaatttcagagctccaacggaaggagaagtctctacaggaggagaacaaggttctgcagaaggagaaagccttactcctgcctgcagctcgcggagaagcagaaagcccagcggcagcaagtgcagcgggaccaaactcaacagcagaccagttcgtcttcctcgtccttcatgataagggaagctgccccaacaacaaatatcagcatcttccctgtggcagcaggcgggagagtggtggaagctgcagcagcgccgccgcaggctcgcgttggactgccaccctggatgcttag

SEQ ID NO: 10 - DNA, 588 bp, Sorghum bicolor
  misc_feature: Ceres Annot ID no. 8642423
atggacatggtttcgcagtccgagcacctgtgctacgtccgctgcacctactgcaacaccgtgctcgcggttggggttccatgcaagaggctgatggacacggtgactgtcaagtgcggccactgcaacaacctctcctacctcagtccacggccccccatggtgcagccgctctcgccgactgatcaccccttggggccattccagtgtcagggaccctgcaatgactgcaggaggaaccaaccgctgccgctggcctcgccgacatcaactgagctcagcccgagaatgcctttcgttgtcaagcccccggagaagaaacaccgcctcccatctgcttacaatcgcttcatgagggaggagattcagcgcatcaaagctgcgaagccagatatccctcacagggaggccttcagcatggctgccaagaattgggcgaagtgcgacccgcgctgctcgacgactgcctctactgccacttccaacagcgctccagaacctagagttgtgcccactcctcaggatagggccaaggagcaagtcattgagagcttcgacatcttcaagcagattgagcgcagcatctag

SEQ ID NO: 11 - DNA, 780 bp, Sorghum bicolor
  misc_feature: Ceres Annot ID no. 8681303
atggggaggggacgagttgagctgaagcggatcgagaacaagatcaaccgccaggtcaccttctccaagcgccgcaacggcctgctcaagaaggcgtacgagctctccgtgctctgcgacgccgaggtcgcgctcatcatcttctccagccgcggcaagctctacgagttcggcagcgccggcataactaaaacactagaaaggtatcaacattgctgctacaacgctcaagattccaatggtgcactctctgaaactcagagctggtaccaggaaatgtcaaaactgagggcaaaatttgaagccttgcagcgcactcagaggcacttgctcggggaggaccttggcccactgagtgttaaggaattgcagcagctagagaaacagctagaatgtgctttgtcacaggcaagacagagaaagacacaacttatgatggagcaagtggaagagcttcgcagaaaggagcgtcacctgggagaaatgaacaggcaactcaaacacaagcttgaagctgaaggttctagcaactacagaaccttgcagcatgccgccgcctggccagctcccggcggcaccatcgtggagcatgacggcgccacgtatcatgttcatccacctgctcactcagttgctatagactgtgaacccactctgcaaattgggtataatacctggtaccctcatcaccagtttctgccttctgatcaggcagccaataatatcccaaggaacgcccccggaggcgagaacaacttcatgctgggatgggttctttga

SEQ ID NO: 12 - DNA, 813 bp, Sorghum bicolor
  misc_feature: Ceres Annot ID no. 8643934
atggggcgcggcaaggtgcagctcaagcggatagagaacaagataaaccggcaggtgaccttctccaagcgccgcaacgggctgctcaagaaggcgcacgagatctccgtcctctgcgacgccgaggtcgccgtcatcgtcttctcccccaagggcaagctctatgagtacgccaccgactcccgcatggacaaaattctcgaacgttatgagcgatattcctatgctgaaaaggctcttatttcagctgaatctgaaagtgagggaaactggtgccacgaatacaggaaactgaaggccaaaattgagaccattcaaaaatgccacaagcacctgatgggagaggatctagagtctttgaatcccaaagagctccaacaactagagcagcagctggagagctcactgaagcacatcagatcaagaaagagccaccttatggctgagtctatttctgaactacagaagaaggagaggtcactgcaggaggagaacaaggctctacagaaggaacttgcggagaggcagaaggcggccgcgagcaggcagcagcagcaggtgcagtgggaccagcagacacagacccaggcccagacaagctcatcatcgtcctccttcatgatgaggcaggatcagcagggtctgccgcctccacaaaacatatgcttcccgccgctgataatcggagagagaggtgaagaggtggctgcggcggcgcagcagcagcagccaccaccagggcaggcgcagcagcaagcgcagctccgcatcgcaggtctgccgccatggatgctgagccacctcaatgcataa

SEQ ID NO: 13 - DNA, 630 bp, Sorghum bicolor
  misc_feature: Ceres Annot ID no. 8669907
atggggcgcggcaagatcgagatcaagcggatcgagaactccaccaaccgccaggtgactttctccaagcgccgcaacgggatcctcaagaaggcgcgggagatcagcgtgctctgcgacgccgaggtcggcgtcgtcatcttctccagcgccggcaagctttacgactactgctcccccaagacatcgctatcaaaaatcttggagaagtaccagaccaactctggaaagatactgtgggatgagaagcacaagagccttagtgccgagattgatcgtataaagaaagagaacgacaccatgcagattgagctcaggcaccttaaaggtgaagatctaaactcactgcaacccaaagacctgatcatgattgaggaggcacttgataatggactgacgaaccagaatgagaaactgatggagcactgggaaaggcgtgttgcaaacaataagatgatggaagatgagaacaaattgctggcctttaaacttcaccagcaggatattgcactgagcggcagcatgagagagcttgagctgggttatcatcctgaccgggacttggcggcccagatgccaatcaccttccgcgtgcagcccagccatcccaacttgcaggacaacaattag

SEQ ID NO: 14 - DNA, 1140 bp, Sorghum bicolor
  misc_feature: Ceres Annot ID no. 8744657
atggcacggcacggtggtggattgcaacgcacgcgcccagagcgagccggccgcgtcagatcacggggcagggggctgggcgcggccggcagtcgcaggcagcgacggccggggcgggcgggcagtgcacggcaccacacggcacggtgccccacgcctttcacggatccggtagctgtctccgtccacgccgcgcaccgcacctcgtcctctccaccccgaattgcacacgcacacaccttgtccttccatcccttgccgcaccaccgcccaccccctcctctcatctgcctctccatttcgtcgtcgtcttcttcttcctcctccttcgatctccacccatccaccgccggcgcggccgggatccgcagctcgagaccgacggtggagcgcggcggcgagcatcagcagccgacgacggcggaggaggaggatccgcgccgccgccgtcccgagacgccgccgccacccacgatggggcgcggcaagatcgagatcaagcggatcgagaacgccaccaaccggcaggtgacctactccaagcgccgcacggggatcatgaagaaggcgcgcgagctcaccgtgctctgcgacgcccaggtcgccatcatcatgttctcctccaccggcaagtaccacgagttctgcagccccgggaccgacatcaagaccatctttgaccgataccagcaggccatcgggaccagcctatggaacgagcagtatgagaatatgcagcgcacgctgagccatctcaaggacatcaatcgcaacctgcgcacagagattaggcaaaggatgggcgaggatctggacactctggagttcgacgagctgcgcggtcttgagcaaaatgttgatgcggctctcaaggagtaccatgtgatcgccacacagactgaaacttacaagaaaaaggtgaagcactcgtacgaggcgtacaagaacctgcagcaggagctgggcatgcgcgaggacccggcgttcgggttcgtggacaacactggcgccggcggttgggacggcgccgcggcggcgctgggcggcggcgccccggacatgtacgccttccgcgtggtgcccagtcagcccaacctgcacggcatggcctatggctcccacgacctccgtcttggctag

SEQ ID NO: 15 - DNA, 975 bp, Sorghum bicolor
  misc_feature: Ceres Annot ID no. 8657974
atgcagagaggcactgccgcaccaccaccaccaccacccatgctcaacatgatgactgatctgagctgccggccgtcggaggtgacagagcagctggcggcgccgacgggctccggcgacaagcaggggaggggcaagatcgagatcaagcgcatcgagaacaccacgaaccggcaggtcaccttctgcaagcgccgcaacggcctcctcaagaaggcgtacgagctctcggtgctctgcgacgccgaggtcgcgctcatcgtcttctccagccgcggccgcctctacgagtacgccaacaacagtgtgaagtccaccattgagaggtacaagaaggccaacagtgacacctccaactctggcacagttgcagaagtcagtgcccagcactaccagcaggagtcctccaagctgcgccagactatcagtagcttgcaaaacgcaaacaggaccatagtgggagattcaatccacaccatgagcctcagggatcttaagcagctggagggcaggctggagaaaggcataagcaagattagagctagaaagaatgagctgttatatgctgaagttgactatatgcaaaaaagggagatggatctgcagactgacaacatgtacctgaggagcaagatcgctgagaataatgaaacggggcagccggcgatgaacatgatgggagtgccatcgacaagcgaatacgagcacatggtcccttttgactcgagaaactttcttcaagtgaacatcatgcagcagcctcagcactactcccatcagctgcaaccaacaaccctgcaactcgggtgtggagtctggatgcggtggcagcggtcgggtgaaggtcaagtagggcgtgccgtgctgacggaccggttgcggtcgagcagcaggagctgcactcgtgctctcaggcaccgagctagtgctcgtccatgtgcacaggcacccgcctgttcttgctgtgagccatga

SEQ ID NO: 16 - DNA, 639 bp, Sorghum bicolor
  misc_feature: Ceres Annot ID no. 8732691
atggggcgcggcaagatcgagatcaagaggatcgagaactccaccaacaggcaagtgaccgtctccaagcgccgggccggactggtcaagaaggccagggagatcggcgtgctctgcgacgccgaggtcggcgtcgtcatcttctccagcggaggcaagctccacgactactgctcgcctaggacctcgttgtccaggatcttggagaagtaccagactaactccgggaagatactgtgggatgagaagcacaagatcctgagtgcagagatcgacagaatcaagaaggagaacgacaacatgcagattcagctcaggcatctgaaaggcgaggacctgaactcgctgcagcccagggagctgatcgccattgaagagggtctccagaatgggcagaccaacatgcgcgagaagcagatggatcactggaggatgcgcaagaggaatgggaagatgctggaggacgagaacaggatgctgtcttttaggatgcatcaacaggctgttgatctgagcggcggcatgagggagctggaaatcggataccatcaggtccagcatgacagggaattcacttcccaaatgccgttcaccttccgggtgcagcccaaccaccccaatctgcaggaagacgagtag

SEQ ID NO: 17 - DNA, 666 bp, Sorghum bicolor
  misc_feature: Ceres Annot ID no. 8031970
atggggcgcggcaagatcgagatcaagcggatcgagaacgccaccaaccggcaggtgacctactccaagcgccgcacggggatcatgaagaaggcgcgcgagctcaccgtgctctgcgacgcccaggtcgccatcatcatgttctcctccaccggcaagtaccacgagttctgcagccccgggaccgacatcaagaccatctttgaccgataccagcaggccatcgggaccagcctatggaacgagcagtatgagaatatgcagcgcacgctgagccatctcaaggacatcaatcgcaacctgcgcacagagattaggcaaaggatgggcgaggatctggacactctggagttcgacgagctgcgcggtcttgagcaaaatgttgatgcggctctcaaggagtaccatgtgatcgccacacagactgaaacttacaagaaaaaggtgaagcactcgtacgaggcgtacaagaacctgcagcaggagctgggcatgcgcgaggacccggcgttcgggttcgtggacaacactggcgccggcggttgggacggcgccgcggcggcgctgggcggcggcgccccggacatgtacgccttccgcgtggtgcccagtcagcccaacctgcacggcatggcctatggctcccacgacctccgtcttggctag

SEQ ID NO: 18 - DNA, 732 bp, Sorghum bicolor
  misc_feature: Ceres Annot ID no. 8655052
atggggagggggcgggttgagctgaagcggatcgagaacaagatcaaccgccaggtcaccttcgccaagcgccgcaacgggctgctcaagaaggcgtacgagctctccgtgctctgcgacgccgaggtcgcgctcatcatcttctccaaccgcggcaagctctacgagttctgcagcggacagagcatcacaaaaacacttgagaggtacgaaaaaagcaattatggaggcccagatactgctgtacagaacaaggagaacgagttagtccagagcagtcgcaatgagtaccttaaactgaaagcaagggtggataatttacaaaggactcagaggaatttgcttggtgaagatctggggtcacttggtatcaaagagcttgagcagctcgagaagcaacttgattcatccttaaggcacataagatccacaaggacacaacatatgcttgatcagctcactgatcttcagaggagggagcaaatgctgtgtgaagcaaataagtgccttagaagaaagctggaggagaccagcaaccaggtgcatggccaagtgtgggagcacggtgccaacttactcggctacgagcggcactcccccccacagcaggccccatcacatgttggcaatggattgttctttcatcccctggaagctgcagcagagccaaccctgcagatcgggttcgctcctgaacatatgaataacttcatgccgacatggctaccctga

SEQ ID NO: 19 - DNA, 822 bp, Sorghum bicolor
  misc_feature: Ceres Annot ID no. 8725895
atgcacatccgagaagagcaagctatgccatcaacagtgacaggcatcatggtgtcgaccctgacttcggcggggctgaagctgaaggagccggcgacgatgtcccctggtggctccgcgtcggttgctgctggatttgggtctgctgcagagaggaacaacggcaggggcaagggcaagactgagatcaagcgcatcgagaacacgaccaacaggcaggtgaccttctgcaagcgccgcaacggcctcctcaagaaggcgtacgagctctccgtgctctgcgacgccgaggtcgcgctcatcgtcttctccagccgcggccgcctctacgagtactccaacaacagcgtgaaggccaccattgagaggtacaagaaggcaaacagtgacaactccagcgctgctggtacgattgcagaggtcacgattcagcactacaagcaggaatctgctaggctgaggcagcagatcactaacttgcagaactccaacagggccctgataggtgatatcacaaccatgagccccaaggacctcaaacacctggagactaggttagacaaagcacttggaaagattagagcaagaaaggaaatggagttgcagaatgacaacttgtacttaaggagcagggttgatgagaatgaaagggcacaacagacagtgaacatgatgggggcgccatcgacaagtgattatcagcagcagggttttattccttatgacccaataaggagcttcctgcagttcaacatcatgcagcagcctcagttttactcccagcaggaggaccggaaagacttcaacctaggtggaagatag

SEQ ID NO: 20 - DNA, 2002 bp, Sorghum bicolor
  misc_feature: Ceres Promoter PD3579
aaactcttcgtcagtgctgatgacagaagcagctgcccttactctagcaaccacggtgctagaagctatgtacatgattgattccactattttaacagataatcaatagttagtactctttctaaacgggtcttagtttgatcatcatcctgatggagaattaaatcctacattcaaattaccagctccaagattcatggtacaactatagcgattcgcaagattaccagaatcatatggctgatcaactagctagataggctctgagtgaattagtttgcaatcaaatctctcttaatagtgcttgttgtcattctgctcatgagcaaaagtgtcctttactttcgacactctcaaatataactattaactctataatggtcctaaccgtaacacgctgttaatcatataggccttgttcagttggcaaaaattttgggttttaacactgtagcatttttgtttttatttgataaacattgtcagatgaactgtgtaattagtttttatttttatgtatatttaatgcaccatacatctgccgtaaaatttgatgggatggaaaatcttgaaaatttttgaaactaaacaaggccatagtttcattgtaaaaaaaaaaacagctaagcaagatggccgagagagccgttgacgcagagcattgaacggcatctctctcggctgctctcgaatgcgctgcctgccggcatcccggaaattgcgtggcggagcggagccgaggcgggctggtctcacacggcacgaaaccgtcccggcacacggcaccacgatttttccttcccctccccctgcccttctttttcctcataaatagccaccccctcctcgcctctttccccccaactcgtcttcgtccctcgtgttgttcggcgtccacggacacagcccgatcccaatccctcttctccgagcctcgtcgatcgcccccttccctcgcttcaaggtacggcgatcgtcctcccgctttcgcttctcccctcccctcctctcgattatgggttattggggctgcgagtcatctttctggcgatttattatggtctcgatctggtggtaactgtggcgatttattatgggagccctcgatctagaagtcgagtactctctctggtaactgtagcgatttgttatgggggctctcgatctagaagccgagtactctctggtaactgtgggacccttgtagggttgggttgttatgattatttgggcttgtgattaggttgtatctgatgcagaatgatgtattgatcgtcctattagattagatggaaacaagtagggtgactctgatttatttatccttgatctcgtttgatgtccctagctaggcctgtgcgtctggttcgtcatactagttttgttgtttttggtgctggttctgatgcccgtccagatcaagtcatatgaaccagctgctgtcttattaaatttggatctgcctgttttaacatatatgttcatatagaattgatatgagctagtatgaactagctgcttgtcttattaaatttggatctgcatgtgttatatgatggatgaaatatgtgcttaagatatatgctgcggttttctgccgaggctgtagcttttgtctgattaaagtgcatcatgcttattcgttgaactctgtggctgtcttaataagaattcatgtttgcctgatgttggagaaaacatacataagaattcatgtttgcctgatgttcgagaaaatatgcatcgacctacttagctattacttgatgcgcatgctttgtcctgttttgtttgatatgcatgcttagaaagattaaaatatatgtggctgctgtttgattcgataattctttagcatctacctgatgagcatgcatgctcttgttattcactgctactgttccttgattctgtgccacctacatgttacatgtttatggttgcttctttttctacttggtgtactactatatgcttacccttttgtttggtttctctgcag

SEQ ID NO: 21 - DNA, 1500 bp, Sorghum bicolor
  misc_feature: Ceres Promoter PD3800
catggaaccaaaggaaacacgtcaaaataatagcaggaaatatatgggcatcagaaagagtgcctgctgcctgtgactactagcttctacacttttcacaatgttgtctgtatctaaccactccatttctttcaaagctttgctggtttcttacacgcatgcaaggaggctattttcctttttcttttctagttcgttcggacgtctcatgtattgcgcaagcaagcacatgcactcatgaaaagctagcaaagacacatgatatggtggcttataaaaaaaagcacatgcattatattgttgtgtagcattttgacatgtatcataattgctactgtggtagtatcttgggtatagatcaccacacaacatttaatttaaaaggcccacggtcgattactagatacatattccttctgtgcaatacaagggattcaaatttgtcctaagtcaaattatcatgagtttgatctaatttaaagaaaagaacatataatatcaaactagtatcattagacacattgttacatacctctctttgctgatgtgatagatattaatactcttctttgtaaattctgtcgtactcagaatatatagtttaacttactttacatttcgggacagagggagtatatatatgttctgttcattctttgcctcgctccctcctttactcatcagtggcaggcaccttttcataatcttatataagtttgcgacacttggtaccgagcacacagcaccatcaccatcactacgtgcaagcaaaggcaacataacttatgtaggacccaattaaagacttaattaatgtagtatcatatttttcatcctactgtacctttttttagggggtgcggggggcttcttgatcactggcctatactgtactgattagtgatgtgtgtttcaccagattgtggctgctggtagtagtgacttggttgctcttgcatgtaacgacatttattgccataatgaatgaagtgctgtatgaaactactcaatgagggcagaggagaacattctaaaaattatttcctagctggaacacgcatttaatttagcacaacattccttccattggtcctaaggttattagggcacacaaatccaaacactacacttggagacttggagagaataatagaacagagagatgcatacaaatcatgcaagctcccagtagagtcctgtggactccttaacatttgctcctggaattgaatatggttaaacaaatgcaggtgcaccatgcatgtcacccctgcctgccatctcatctcatccacagtgcctgcccctgcatgccctccttcctttgctttccctcccaaaggacacctccaagctccatttaaataccacccctccctccctcacttgtgaccactactgcactacactactccaaaacgacctcaagccagcactcaacctaggtagctcacagccacagcagctaaagcctattagctcactcgtgctcatcttgcc

SEQ ID NO: 22 - DNA, 980 bp, Sorghum bicolor
  misc_feature: Ceres Promoter Annt 8643934
aaacgatgcaagcctcacgttgtgcagcacaacaccatcgacgactgcccttttatttaatttcgtgaccaaacggcccaaatgccgactgcatttttttctactctctactctcatcttcaataaactacattatgtatgtgtccatattaatttattagtttgagcatatttatatacttaatttaagatacttcaaatcttaatatgcgaaaaaattcagtacatctcatactttttaatataattactaaaatatcacttcaatagtatataaaataaatatatactatatttatacctacatatttaacatacgtactagtatgaccatacatatactctatcaacacatactttatcgggctatatacgtcataaatatatatatatatatatatatatatacacacgcgcgcgcgacgttgaaaaaaaactaggcctgcatgtgtttttttttggttgttcgaccgtacgaggagctcgctctcacgtagcccgatggaacccccatttattctcctttttcaacgcattttttcttgtacatatattagttaattaattaaggtcgagtaataagtagtaccactgggggaagagaaagagagagagaagcacaggcgctgcccgcagcagcgcgtcagcgcccgggacgacgagagagagagaggctgacaaggtgggcccgtgcgggccttgaccaatcggagttcgacagcagcctgcccccaaaaccacactcgctctcctcccctcgcgccgcggccgtcgcctcccctccctccaccgatcgatccctctcctcctcctcacatcccaccccccaccttctctttaaagctacctagctacctacctacctgccgcctcgccggcggccgctagctgccagtcgccaccgccgccgcatcgatcgatcggaacggaaggagctagctagcgcagcaagcgcccatcagcaagcagatcggagcaag

SEQ ID NO: 23 - DNA, 1000 bp, Sorghum bicolor
  misc_feature: Ceres Promoter Annt 8632648
ccggaccgcacaaacagaccgagccaggcgacctgttgctcccacttccccgttttgcccaaacattttcgtctcgttccgtcccgtagaccggcgccccccatccccccacgccgcagctttcttcacgtgcacggtgcacggcacgctgcgcttaaaaaggaaacaaaaacaaccagccgcaagctccaaaagatctgaaatctccaatgcttgggcacccggcaccactgggcaaatctgaaataatactaaaatccagtccacaaacaatctcgataccaaaatcgacaacaaagaatctagtgagatttcttaaatatatatatatacacactaatcaggactagtataggagtagtgcatattttttatatatagaaaaacaaaaacagataatagctgccaacaacttgtcgtgccaggctacgctgggagagagaagccggtttcgaccgtacgaggaaggaccttggccctggcggcgggcccatgcgatgagagatgcggtggggccctccgggcccgggcatggggcatcggccaatggctgttcgacagcggcggctcggaaaccatcccggtttcgcgataccccttcctcccctccgatccgtcgcggcagcgcgcatcaccgctttaaatccgccccctcccggcgcctccctcctcccggccggtccccctcaccttctctccctctcctctctctccagcctccaccgccgcagctagctagctgtgacgtcatgcactcgccggcgccatagcgcgccagctcctactatctacaactgtaggcttagttatcctgtcaatcaagcctctcgtaaggaacaaggaaggtagctagatagttttatagctgctgtcgtcgtcgtcggcggcggcggaagcgacgtacctgttcttagaggataggataggttagcagagagggtcagctagcgaggattttggttgagatcaggagggggaggaggaggaggaggcggcggcg

SEQ ID NO: 24 - DNA, 1500 bp, Sorghum bicolor
  misc_feature: Ceres Promoter Annt 8657974
ccaactctgatgtgtccacaatgccaccagcaacctgctatgtatcttgaaactgactgttcgttgataacatggattagagcagtcagtggagtggtagcttcacaacgacacattattgatcgatgaggatgtgcgtccactgtacatacatccatgcatattagtagtggcccaggtgagtgatggagttgggagggggcggtgggcaaggaatatgcatccgatccaccacattatgtaagggccccggttgggtttgggtcccattaattcatcccatgaggctatctccaacagggagacccatttgggacccaaacctaaaatgggtctccaacacaatacctatagcctccaacagagtacccatacagaagacctattttgggtatcaggagaggcataacccaaatttgggtatcctctctcctcgagacccatttgcagagagtgttgtcttttaggtcttgttgttggagaagactaaaaataggtatggaaccttttacctgtagcgctatccaaaggacaaatgggtcttgtattttgggtgacgattgttggagatagtctgagagcaacagaatgggagctgaaaataattgggatgctgtagctggatagtacttagcccatcatcccctgcatcatatatacacatatatgtgccaaacagaaatgggacagtgtttgtgcggtgtcacatgcggatagggcacacaatgttcctcatctatacatgcctgccttgatcacaagatattgaagaaaaaaagtgaagatgaaacaaaaagaatatgggataacagacactacagaattaacattttattagttcactagctcacaaaaaaagtaaatcattttatatgtataagtttgaattagaagatcaattttttattaatcattgtaaaaaactacagactttattataccggctgcctcatactatttaatcagtgagagtctgtaaaagaattgaaccgataaaaaaaggttcagaagcaaaactattgcaaattagttctaccaagccaatgagaattattgcaattcaggggggctagctaaagatgcactagcattgttgttacagttggggcttgctatatatgaacttctatcgttctctttcactctgcttggtgaattaaagtgcaaaaccttaaagagttaagcaagaaatagccagccttgctgcaccatatgcaaaacggtttttgtggtaattgtgtttgtgtgcctgacatatgcatgctgactgcagaggtagatggagatctgacaccaagcaagcacacacacacatacaagagggatcaatcaaaaaagtggtaaatcaaaaaggcttgtgcaagtcatcagaagcccagggggaacaaacaaaacaatcagcacaggccataggaattggccacagcccacagctgcacctgcatacagctgctgctaactgcatcacctcacataccttcccctctcttccttcctcag

SEQ ID NO: 25 - DNA, 1000 bp, Sorghum bicolor
  misc_feature: Ceres Promoter Annt 8681303
ctatctaaatttctcatctctcatctcatatcatgtaatggcagcatgcatggatctcatgatctcaatgatgtgtgttgtcgtcgtcatcgccgtcccatacatgcctacagaaatctggcacaaaggcgggcggctcacgtactagcatatacgctacaggcagcacacatgcaagtcgtactagcatgcaaccaagggaaaattggcaattggggttatggaatcaaacagggatctatatttttgggcagcctgcctccaccggtcggatcggagaagggttggatcatatcggaggcgcgcggcgtggcccaaagcgaaagcaacggcgcagggctgcagggttgagagcctgctgggacccaggaggtgtgtgaggtgcatggcgacctgcatggtttgggttaggcctaggaggttctctctccaatgggtagctcgcaccctttggccgccgttctcgtgtatgccatatgccatcatgctatgcttcgcgccgtgtatatttgtggcgtgggagccgccgcatcggagcgcccccgtttcggcacgggtctcccagttagggtaaaccaggggcagtgggtaaaactgcccagctccactccaaatttaccctccggcctctctcctttaatttaaagctagctctgagagatagatgggagtgcagagtgggggagatagatggatggatggagacgcgtgaaaaagatgcgatgcgagagaagagaccggcagcgtgctacagggcagccacacagtgacgctgccctttttcccggttctctccactgatattccgctcctgtcctgccccggcgacccattatatcgtcgcgcacaatgcaaaacttagcacttgtgggcggccttgttttgtggggagtcgtcgcctagctagcagcggataagcaggcagcagcagaagagcgagcagagagagcggctgagtgagagagagaacagcgagctctcggaggagaaggagatcgtc

SEQ ID NO: 26 - DNA, 1000 bp, Sorghum bicolor
  misc_feature: Ceres Promoter Annt 8732691
ctgatcgtgaatcttggtataggcacttaagtggaggtatgttgcaatgtctacacaaaatcaccgtcgcttgcaataacaaatacctatgatttaactatttctatatattgttgatcgtgaatcttgctacaggcacttaagtggatttctcatcctaagtatatgtctacatggaaaagaccaaaaaccacacacatttatacagcgagtataacaattttgtctatgtttttgtgaaagttttttcacaaaatgtagtattacccatgcgacacacactaatatttacatatatatatacatactcgaacgtgtgataggatggtacagagatatggtagaacttatttgtggatttacagtgcacgaaagaaatggatatcggagatttcttcctgccgatattaaatattatcttagtcgtatcacggaaatgtctataaagtagaatttatgagcctacaacgtcgtgctctctgtggctgtgttttatttcacaaactttgcttaactttgctttttaagataaatatcactttaaaattggtatttaagttttattataatattattattttattatgcgatccgtatattttaaataaaaatattagttattccctagcaacgtataggcacgctacatagtaaaagaaaaaagaaaatgattaaaaaaacataggccccaccgagcagcacaggctgcactacgtgcgaacgtaatcaacacagccacgctagccgtttccatttccggccctgcaatcctgcaccggcgcgaaagccccctcacggcctcacctccatcctccactccgtcgatggccgcctctcctttactcctcagtgctcacgccgtccacagtccactagtggcatcggctcccacataaaagatccacaccacttgagcagaaggctggtagtggaagggtaggctcttgcttgagctgagctcttgctgcctgtggatctctttgggagtggtgaacggagg

SEQ ID NO: 27 - DNA, 1000 bp, Sorghum bicolor
  misc_feature: Ceres Promoter Annt 8031970
catttgtaatgcatgtaagctgtggtccagaccttgtttggaacaaagttaaataaaaatacatgcatgaaacatgtacaatctaatttcgtttaattagattccattataaaatatacccactacattacttgacaacaacaattattcatatatatttcttctacaaacttaagaggcaatataaatgatgacccaataatatatggtgttgtgtagagtggacggggccagtgacgtgacgatgtaggtataaacaaataagagagctagctagctagcaggctactacggtgcatgctttgctttcagtccagtgcaaattaaaggggtgcatgcatgcatcagtgtggaggccaacaagatcgagaagaagcagtgcccaagaagaggatccggaagccaaaaccgtgctaaccgttgtgccaaaagccgccacggcagaccgaccgaccgaccacgaccagaccgatcgtcgacggatgcatggcacggcacggtggtggattgcaacgcacgcgcccagagcgagccggccgcgtcagatcacggggcagggggctgggcgcggccggcagtcgcaggcagcgacggccggggcgggcgggcagtgcacggcaccacacggcacggtgccccacgcctttcacggatccggtagctgtctccgtccacgccgcgcaccgcacctcgtcctctccaccccgaattgcacacgcacacaccttgtccttccatcccttgccgcaccaccgcccaccccctcctgcttattaccaccaccggctctctcttgtgctgtgctcagctcatctgcctctccatttcgtcgtcgtcttcttcttcctcctccttcgatctccacccatccaccgccggcgcggccgggatccgcagctcgagaccgacggtggagcgcggcggcgagcatcagcagccgacgacggcggaggaggaggatccgcgccgccgccgtcccgagacgccgccgccacccacg

SEQ ID NO: 28 - DNA, 890 bp, Sorghum bicolor
  misc_feature: Ceres Promoter Annt 8669907
ccgggaagatagccaactacccgtcgaaacaacaatttgcactgcatttccgatttctttattagttgaaaatagactattagtaaagaaccattttataactatgtaaaattagggtctatttagcgggtttttttttcaagtgctccttggccagagtacttgagaaaaagtcactttgtaaattcggttcatatcttaaaaatagcttcgactcctttactactcatcagagccttctaaagtgtttagttccgcttctttatggaagatgtcatagaaggtagagtcgtgctaaatagactttgaaaatactaaaaaggcattggtgaaaatatttttcttttcgatgataagtctattgtgatattgtgaaaactgtagacaatttatttgttttgtcaaataggattttgttaacgctgatgaaatcgcgatatcatatcataaatttgacaaagggtctacgtttttacatctactgagaccccttaaagtcctaccgagagaaacgtcgacaagacacgacatgggaagtacacaacgccattaccgcatatagtaccctccctctgctttcggctttgccatcaactgcaaccggacagcgaggtgaagtcaagttgccccgcggacgcaaagcatacaagtcatttgccgtttccggcgcgcgcctctgaacaaacccgaaagccccctcatcaccattcctcttcctgccatggcgatcccctttactcctcccctcccctcccaccaccaccactgcccctcccaactacaccacaccggcaccaccaccaaacgagcagctccaggctcttgctcaagaaggggagaagaggcgagcctttattgggaagttgctggaggagagaaggggaggaagg

SEQ ID NO: 29 - DNA, 1000 bp, Sorghum bicolor
  misc_feature: Ceres Promoter Annt 8642422
aatcagtctcagctgcaaacactccatacatgtgctaaatatatatagttgcatttagcaccccagcatctctggcacaaaaaagcaacgagtttccaaatatataacgtacgactaactatatattcaactattgtgcgagggtatttgcaacttattagactaatccttggattttctatcataagcgcgtacatttacaagggaagtgtgaatgaactgtaccatcattacctataagcctattccctattcattgttccatgtcacactctttaaacatatatatattgtcttctagagagcaaatgctgcttacaaattacaatgtatgatacaggcattgtttcttagaaatcaaacgtcaattaattgatgacagtggcctcgatcgctcataacatgtgaatgcggttttcaaaaattgcttcttgtgcctagatatatcttggatctcactctatgaagacactatagatatatagtatgctgagttggtgcacaaaagcatcagcaaatttttataatatgtattgcacctttatacaagaaacgtatctaataaattggagtcaaattaacatattttcatggtgtgctgacatttggagcagcaactagaactccagcttttgttgtatatatatgaaaactagatgtaaagcataagctaattatgtttgttgggataataccaacgaaacttccttcttaattagtgaattatcactgtatgcaagtgttttatttttaatttgccgtgcagcatttctatttttgtagttaattagctttcaccagatcatggcactgtggatgtccactaaacagaatttaatacaatccttacaaaattaattaaggatttttaaccctcagaattatatatttcttttgtcaaaagagcaaataaaaggagcatcaaggcgaaatcaaagaacaatcttattttttttctaaattctgccattgatcagtttgattgccttgtgccaacagc

SEQ ID NO: 30 - DNA, 20 bp, Artificial sequence
  UAS of HAP1 of Saccharomyces cerevisiae
agcacggacttatcggtcgg

SEQ ID NO: 31 - DNA, 22 bp, Artificial sequence
  UAS of HAP1 of Saccharomyces cerevisiae
gcagcggtattaacgggattac

SEQ ID NO: 32 - DNA, 20 bp, Artificial sequence
  UAS of LexA of Escherichia coli
tactgtatatatatacagta

SEQ ID NO: 33 - DNA, 20 bp, Artificial sequence
  UAS of Lac Operon of Escherichia coli
aattgtgagcgctcacaatt

SEQ ID NO: 34 - DNA, 18 bp, Artificial sequence
  UAS of ArgR of Escherichia coli
wntgaatwwwwattcanw

SEQ ID NO: 35 - DNA, 17 bp, Artificial sequence
  UAS of AraC of Escherichia coli
tatggataaaaatgcta

SEQ ID NO: 36 - DNA, 188 bp, Artificial sequence
  UAS of Gal4 of Saccharomyces cerevisiae
attatcggagtcagagctgccgcttcggtagagtcgaatccgacagcgggtgacagccctccgagaccggaagactctcctccgtagcggaccgcactgctccgatccggaagactctcctccgcgacgggtgacagccctccgaagcggaccgcactgctccgaacgcggacaactgttgaccgtga

SEQ ID NO: 37 - DNA, 124 bp, Artificial sequence
  UAS of HAP1 of Saccharomyces cerevisiae
accgcgggtataccggtgatacatgtacggacgtatcggtggtagtccagagactcggtgttatcggaagtattgaaccacgggcttatcgggcctaagatcgtcggagctagcggcagtactc

SEQ ID NO: 38 - protein, 881 aa, Saccharomyces cerevisiae
  misc_feature: GAL4
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSIDSAAHHDNSTIPLDFMPRDALHGFDWSEEDDMSDGLPFLKTDPNNNGFFGDGSLLCILRSIGFKPENYTNSNVNRLPTMITDRYTLASRSTTSRLLQSYLNNFHPYCPIVHSPTLMMLYNNQIEIASKDQWQILFNCILAIGAWCIEGESTDIDVFYYQNAKSHLTSKVFESGSIILVTALHLLSRYTQWRQKTNTSYNFHSFSIRMAISLGLNRDLPSSFSDSSILEQRRRIWWSVYSWEIQLSLLYGRSIQLSQNTISFPSSVDDVQRTTTGPTIYHGIIETARLLQVFTKIYELDKTVTAEKSPICAKKCLMICNEIEEVSRQAPKFLQMDISTTALTNLLKEHPWLSFTRFELKWKQLSLIIYVLRDFFTNFTQKKSQLEQDQNDHQSYEVKRCSIMLSDAAQRTVMSVSSYMDNHNVTPYFAWNCSYYLFNAVLVPIKTLLSNSKSNAENNETAQLLQQINTVLMLLKKLATFKIQTCEKYIQVLEEVCAPFLLSQCAIPLPHISYNNSNGSAIKNIVGSATIAQYPTLPEENVNNISVKYVSPGSVGPSPVPLKSGASFSDLVKLLSNRPPSRNSPVTIPRSTPSHRSVTPFLGQQQQLQSLVPLTPSALFGGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE